(19) United States
(12) Patent Application Publication
(10) Pub. No.: US 2022/0387365 A1
(43) Pub. Date: Dec. 8, 2022

(54) TUCARESOL DERIVATIVES AND USES THEREOF

(71) Applicant: BeyondSpring Pharmaceuticals, Inc., New York, NY (US)

(72) Inventors: Chengjiang He, New York, NY (US); Lan Huang, New York, NY (US); James R. Tonra, New York, NY (US)

(21) Appl. No.: 17/775,851

(22) PCT Filed: Nov. 9, 2020
(86) PCT No.: PCT/US2020/059722

§ 371 Date: May 10, 2022

(30) Foreign Application Priority Data
Nov. 11, 2019 (CN) 201911094713.8

(51) Int. Cl. A61K 31/198; A61K 39/395; A61K 45/06; A61K 31/496; A61P 35/00; A61K 31/405; A61K 31/4172; A61K 31/7034; A61K 31/235; A61K 31/24; C07C 235/84; C07D 209/20; C07C 323/59; C07D 233/64; C07H 15/18; C07C 69/94; C07C 251/48

(52) U.S. Cl. CPC A61K 31/198; A61K 39/3955; A61K 45/06; A61K 31/496; A61P 35/00; A61K 31/405; A61K 31/4172; A61K 31/7034; A61K 31/235; A61K 31/24; C07C 235/84; C07D 209/20; C07C 323/59; C07D 233/64; C07H 15/18; C07C 69/94; C07C 251/48

(57) ABSTRACT

Disclosed herein are tucaresol derivative compound and composition containing the same. Also disclosed herein are methods of enhancing immune response in a subject by administering the tucaresol derivative compound or by co-administering the tucaresol derivative compound and one or more additional agents. Also disclosed herein are use of the tucaresol derivative compound and composition containing the same in the manufacture of a medicament for treating cancer or enhancing immune response in a subject.

### CROSS REFERENCE TO RELATED APPLICATIONS

This patent application is a U.S. National Phase Application of PCT/US2020/059722 filed on Nov. 9, 2020, which claims the benefit of priority to Chinese Application No. 201911094713.8, filed Nov. 11, 2019. All of the foregoing applications are fully incorporated herein by reference in their entireties for all purposes.

### BACKGROUND

### Field

The present disclosure relates to the field of chemistry and medicine. More particularly, the present disclosure relates to Tucaresol derivative compounds and methods of making and using the same.

### Description of the Related Art

Human cancers harbor numerous genetic and epigenetic alterations, generating neoantigens potentially recognizable by the immune system (Sjoblom et al, 2006). The adaptive immune system, comprised of T and B lymphocytes, has powerful anti-cancer potential, with a broad capacity and exquisite specificity to respond to diverse tumor antigens.

A continuing need exists to develop immunization models that enhance cell responses against diseases such as, but not limited to, infectious diseases or cancer. Thus, efforts to develop immune modulator and specific immune checkpoint inhibitors have begun to provide new immunotherapeutic approaches for treating cancer, such as the development of an antibody, ipilimumab, that binds to and inhibits Cytotoxic T-Lymphocyte Antigen-4 (CTLA-4) for the treatment of patients with advanced melanoma. In addition, nivolumab and pembrolizumab, which are anti-PD-1 antibodies, have been approved for treating melanoma, NSCLC and renal cancer, but patients had only limited response to these therapies. There exists a particular need for developing effective therapeutic agents that can boost immune responses against cancer cells in cancer patients.

### SUMMARY

Some embodiments relate to a compound having a structure of formula

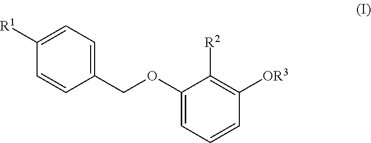

wherein:
R¹ is —COOH, —COOR^{1a}, —COO(CH₂)ₘC(O)NR^{1a}R^{2a}, —CONHR^{1b}, —COR⁴, or —CONH(CH₂)ₘCOOR^{2b};
R² is —CH(O) or —CH(═NOR^{1a});
R³ is H, —C(O)R^{1a}, optionally substituted —C₁₋₁₀ alkyl, optionally substituted C₂₋₁₀alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl;
R⁴ is an amino acid residue attached through an N-terminal amine;
each R^{1a}, R^{2a}, R^{1b} and R^{2b} are independently selected from —H, halogen —OH, —COOH, —COO(C₁₋₄alkyl), optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl; and
m is an integer between 0 to 3.

Some embodiments relate to a pharmaceutical composition comprising a compound described herein and at least a pharmaceutically acceptable carrier or excipient. Some embodiments relate to a pharmaceutical composition comprising a therapeutically effective amount of a compound described herein and a pharmaceutically acceptable excipient.

Some embodiments relate to a method of enhancing immune response in a subject, comprising administering the compound or composition described herein.

Some embodiments relate to a method of treating cancer, comprising administering the compound or composition described herein to a subject in need thereof.

Some embodiments relate to a method of enhancing immune response in a cancer patient, comprising administering the compound or composition described herein.

Some embodiments relate to methods for stimulating an immune response, enhancing immunogenicity of an immunogen, and methods of treating an infection, an autoimmune disease, an allergy, using the compound or composition described herein.

Some embodiments relate to methods of providing co-stimulation of T-cell activation against cancer by co-administering a compound of formula (I), with one or more immune checkpoint inhibitor. Some embodiments relate to methods of providing co-stimulation of natural killer cells against cancer by co-administering a compound of formula (I), with one or more immune checkpoint inhibitor.

Some embodiments relate to use of the compound or composition described herein in the manufacture of a medicament for treating cancer in a subject.

Some embodiments relate to use of the compound or composition described herein in the manufacture of a medicament for enhancing immune response in a subject.

Some embodiments relate to use of the compound or composition described herein in the manufacture of a medicament for enhancing immune response in a cancer patient.

### DESCRIPTION OF DRAWINGS

FIGS. 1A-1D are graphs showing the plasma tucaresol levels following IV (3 mg/kg) or PO (10 mg/kg) dosing of tucaresol, Compound C01, Compound C02-1 and Compound C02, respectively.

FIG. 2A and FIG. 2B are graphs demonstrating the results of tucaresol and compound C02 efficacy study in murine liver cancer model: FIG. 2A shows the treatment results of group 1 to group 5 (once daily dosing for 5 days), and FIG. 2B shows the results of group 6 to group 10 (every other day dosing for 11 doses).

FIG. 3A and FIG. 3B are graphs showing the inhibition activity of tucaresol, anti-PD1, and anti-CTLA4 groups at various doses in MC38 Murine Colorectal Cancer model.

FIG. 4A and FIG. 4B are graphs respectively showing the tumor growth and survival rate of Compound C02 and anti-PD1 combination at various doses in MC38 Murine colorectal cancer model.

FIG. 5A and FIG. 5B are graphs respectively showing the tumor growth over time of Compound C02 and anti-PD1 combinations over time.

### DETAILED DESCRIPTION OF THE PREFERRED EMBODIMENT

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable salts can also be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺and Ca²⁺and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—, (CH₃)₂CHCH₂—, and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms, for example, one, two, three, four or five heteroatoms. The heteroalkyl group may contain 1-2, 1-3 or 1-4 heteroatoms. By way of example only, “C₁₋₄heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. The heteroaryl may contain one or more heteroatoms, for example, one, two, three, four or five heteroatoms. The heteroaryl may contain 1-2, 1-3 or 1-4 heteroatoms. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl may contain one or more heteroatoms, for example, one, two, three, four or five heteroatoms. The heterocyclyl may contain 1-2, 1-3 or 1-4 heteroatoms. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline. An “optionally substituted 5-6 membered heterocyclyl” may contain one oxygen heteroatom, optionally substituted with up to four substituents each independently selected from —OH, —OC(O)CH₃, —CH₂OH, —CH₂OC(O)CH₃, —(C₂₋₃ alkylene)-OH, and —(C₂₋₃ alkylene)-OC(O)CH₃.

A “(heterocyclyl)alkyl” is a heterocyclyl group connected, as a substituent, via an alkylene group. Examples include, but are not limited to, imidazolinylmethyl and indolinylethyl.

A “monosaccharide” or “monosaccharide ring” refers to a single sugar residue. The monosaccharide may have a five- or six-membered carbon backbone. Non-limiting examples of “5-6 membered monosaccharide ring” include: glucose (e.g., D-glucose), deoxyglucose (e.g., 2-deoxy-D-glucose), galactose, fructose, ribose (e.g., D-ribose), and deoxyribose (e.g., 2-deoxy-D-ribose). An “amino monosaccharide” refers to an amino sugar wherein the sugar backbone is a monosaccharide.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “cyano”group refers to a “—CN” group.

A “cyanato”group refers to an “—OCN” group.

An “isocyanato”group refers to a “—NCO” group.

A “thiocyanato”group refers to a “—SCN” group.

An “isothiocyanato”group refers to an “—NCS” group.

A “sulfinyl”group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “sulfonyl”group refers to an “—SO₂NR_{A}R_{B}” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “S-sulfonamido” group refers to a “—SO₂NR_{A}R_{B}” group in which R_{A}and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-sulfonamido” group refers to a “—N(R_{A})SO₂R_{B}” group in which R_{A}and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “O-carbamyl” group refers to a “—OC(═O)NR_{A}R_{B}” group in which R_{A}and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “O-thiocarbamyr”group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “amino” group refers to a “—NR_{A} R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more subsitutents independently selected from C₁₋₆ alkyl (optionally substituted with —OH or O-carboxy), C₁₋₆ alkenyl, C₁₋₆ alkynyl, C₁₋₆ heteroalkyl, C-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Unless otherwise indicated, wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

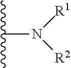

and R¹ and R² are defined as selected from the group consisting of hydrogen and alkyl, or R¹ and R² together with the nitrogen to which they are attached form a heteroaryl, it is meant that R¹ and R² can be selected from hydrogen or alkyl, or alternatively, the substructure has structure:

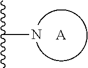

where ring A is a heteroaryl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

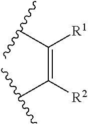

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

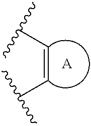

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

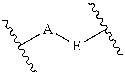

includes the substituent being oriented such that the A is attached at the leftmost attachment point of the molecule as well as the case in which A is attached at the rightmost attachment point of the molecule.

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Compounds

Some embodiments relate to a compound having a structure of formula

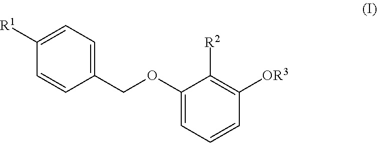

wherein: R¹is —COOH, —COOR^{1a},—COO(CH₂)ₘC(O)NR^{1a}R^{2a}, —CONHR^{1b}, —COR⁴, or —CONH(CH₂)ₘCOOR^{2b};
R² is —CH(O) or —CH(═NOR^{1a});
R³ is H, —C(O)R^{1a}, optionally substituted C₁₋₁₀ alkyl, optionally substituted C₂₋₁₀ alkenyl, optionally substituted C₁₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl;

R⁴ is an amino acid residue attached through an N-terminal amine;
each R^{1a}, R^{2a}, R^{1b} and R^{2b} are independently selected from —H, halogen, —OH, —COOH, —COO(C₁₋₄alkyl), optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl; and
m is an integer between 0 to 3.

In some embodiments, any one or more of R^{1a}, R^{2a}, R^{1b} and R^{2b}, if present, are independently a 5-6 membered heterocyclyl containing one oxygen heteroatom, substituted with up to four substituents each independently selected from —OH, —OC(O)CH₃, —CH₂OH, —CH₂OC(O)CH₃, —(C₂₋₃ alkylene)-OH, and —(C₂₋₃alkylene)-OC(O)CH₃. In some embodiments, any one or more of R^{1a}, R^{2a}, R^{1b} and R^{2b}, if present, are independently an optionally substituted 5-6 membered monosaccharide ring. In some embodiments, any one or more of R^{1a}, R^{2a}, R^{1b} and R^{2b}, if present, are independently selected from the group consisting of glucose, galactose, deoxyglucose, fructose, ribose, and deoxyribose. In some embodiments, any one or more of R^{1a}, R^{2a}, R^{1b} and R^{2b}, if present, are deoxyglucose. In some embodiments, any one or more of R^{1a}, R^{2a}, R^{1b} and R^{2b}, if present, are independently

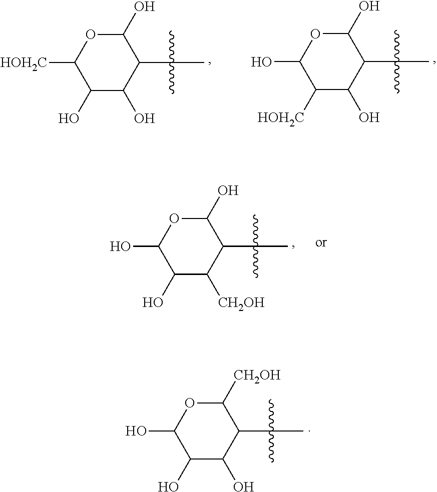 [SMILES: *C1C(O)OC(CO)C(O)C1O ; *C1C(O)OC(O)C(CO)C1O ; *C1C(O)OC(O)C(O)C1CO ; *C1C(CO)OC(O)C(O)C1O]

In some embodiments, any one or

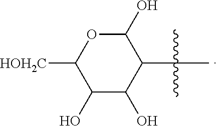 [SMILES: *C1C(O)OC(CO)C(O)C1O]

more of R^{1a}, R^{2a}, R^{1b} and R^{2b}, if present, are In some embodiments, any one or more of R^{1a}, R^{2a}, R^{1b} and R^{2b}, if present, are independently a 5-6 membered monosaccharide ring, selected from the group consisting of D-glucose, 2-deoxy-D-glucose, D-ribose, and 2-deoxy-D-ribose.

In some embodiments, one, two, or three H atoms in —(CH₂)ₘ— are each independently replaced with halogen, —OH, —COOH, —COO(C₁₋₄ alkyl), optionally substituted C₁₋₃₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, and optionally substituted 5-10 membered heteroaryl.

In some embodiments, the compound has a structure of formula (II)

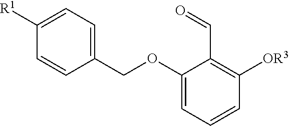

or a pharmaceutically acceptable salt thereof.

In some embodiments, the compound has a structure of formula (III)

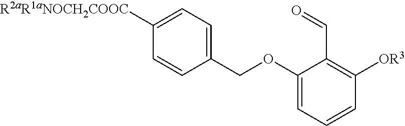

or a pharmaceutically acceptable salt thereof.

In some embodiments, R¹ is —CONHR^{1b} and R^{1b} is an optionally substituted 3-8 membered heterocyclyl, or an optionally substituted 5-6 membered monosaccharide ring. In some embodiments, R¹ is —CONHR^{1b} and R^{1b} is an optionally substituted 3-8 membered heterocyclyl. In some embodiments, R^{1b} is a 5-6 membered heterocyclyl containing one oxygen heteroatom, substituted with up to four substituents each independently selected from —OH, —OC(O)CH₃, —CH₂OH, —CH₂OC(O)CH_{3,}, —(C₂₋₃ alkylene)-OH, and —(C₂₋₃ alkylene)-OC(O)CH₃. In some embodiments, R^{1b} is an optionally substituted 5-6 membered monosaccharide ring. In some embodiments, R^{1b} is a 5-6 membered monosaccharide ring, selected from the group consisting of D-glucose, 2-deoxy-D-glucose, D-ribose, and 2-deoxy-D-ribose. In some embodiments, R^{1b} is selected from the group consisting of glucose, galactose, deoxyglucose, fructose, ribose, and deoxyribose. In

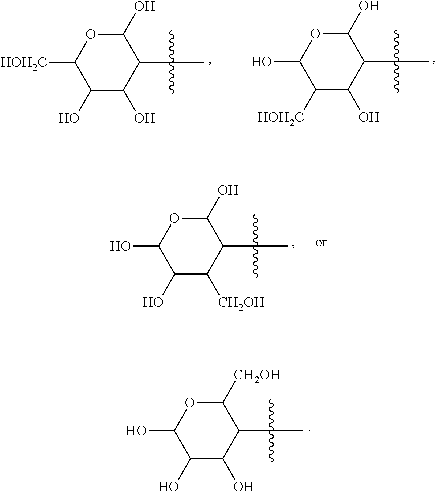 [SMILES: *C1C(O)OC(CO)C(O)C1O ; *C1C(O)OC(O)C(CO)C1O ; *C1C(O)OC(O)C(O)C1CO ; *C1C(CO)OC(O)C(O)C1O]

some embodiments, R^{1b} is a deoxyglucose. In some embodiment, R^{1b} is

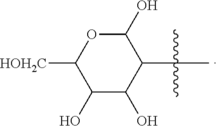 [SMILES: *C1C(O)OC(CO)C(O)C1O]

In some embodiments, R^{1b} is

In some embodiments, R² is —CH(═NOR¹a). In some embodiments, R² is —CH(═NOH).

In some embodiments, R¹a is a C₁₋₆ alkyl. In some embodiments, R^{1a} is ethyl, butyl, cetyl, decyl or dodecyl.

In some embodiments, R¹ is —COO(CH₂)ₘ C(O)NR^{1a}R^{2a}; and one of R^{1a}and R^{2a} is a 5-6 membered heterocyclyl containing one oxygen heteroatom, substituted with up to four substituents each independently selected from —OH, —OC(O)CH₃, —CH₂OH, —CH₂OC(O)CH₃ , —(C₂₋₃ alkylene)-OH, and —(C₂₋₃ alkylene)-OC(O)CH₃. In some embodiments, R¹ is —COO(CH₂)ₘC(O)NR^{1a}R^{2a}; and one of R^{1a}and R^{2a} is an optionally substituted 5-6 membered monosaccharide ring. In some embodiments, the 5-6 membered monosaccharide ring is selected from the group consisting of D-glucose, 2-deoxy-D-glucose, D-ribose, and 2-deoxy-D-ribose. In some embodiments, R¹ is —COO(CH₂)ₘC(O)NR^{1a}R^{2a}; and one of R^{1a} and R^{2a} is selected from the group consisting of glucose, galactose, deoxyglucose, fructose, ribose, and deoxyribose. In some embodiments, R¹ is —COO(CH₂)ₘC(O)NR^{1a}R^{2a}; and one of R^{1a}and R^{2a} is deoxyglucose. In some embodiments, R¹

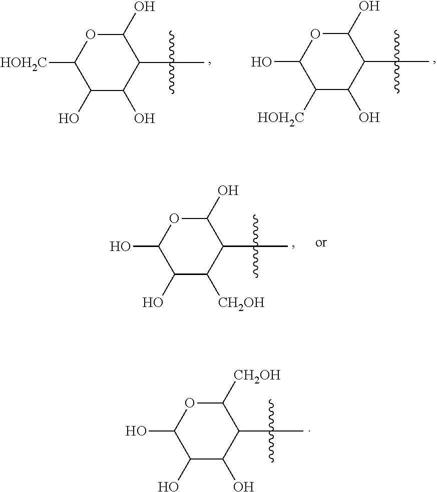 [SMILES: *C1C(O)OC(CO)C(O)C1O ; *C1C(O)OC(O)C(CO)C1O ; *C1C(O)OC(O)C(O)C1CO ; *C1C(CO)OC(O)C(O)C1O]

is —COO(CH₂)ₘC(O)NR^{1a}R^{2a}; and one of R^{1a}and R^{2a} is In some embodiments, R¹ is

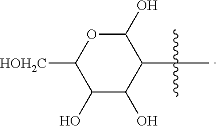 [SMILES: *C1C(O)OC(CO)C(O)C1O]

—COO(CH₂)ₘC(O)NR^{1a}R^{2a}; and one of R^{1a}and R^{2a} is In some embodiments, the other of R^{1a} and R^{2a}is H. In some embodiments, m is 0, 1, or 2. In some embodiments, m is 0. In some embodiments, m is 1. In some embodiments, m is 2.

In some embodiments, R¹ is—COR⁴. In some embodiments, R¹ is —CONHCH(COOH)(CH₂)ₙR⁵, n is an integer between 0 to 5, and R⁵ is selected from—H, —OH, —COOH, —COO(C₁₋₄alkyl),—NHC(═NH)NH_{2,} optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl. In some embodiments, R⁵ is an optionally substituted 3-8 membered heterocyclyl, or an optionally substituted 5-6 membered monosaccharide ring. In some embodiments, R⁵ is an optionally substituted 3-8 membered heterocyclyl. In some embodiments, R⁵ is a 5-6 membered heterocyclyl containing one oxygen heteroatom, substituted with up to four substituents each independently selected from —OH, —OC(O)CH₃, —CH₂OH, —CH₂OC(O)CH₃, —(C₂₋₃ alkylene)—OH, and —(C₂₋₃ alkylene)-OC(O)CH_{3.} In some embodiments, R⁵ is an optionally substituted 5-6 membered monosaccharide ring. In some embodiments, R⁵ is a 5-6 membered monosaccharide ring, selected from the group consisting of D-glucose, 2-deoxy-D-glucose, D-ribose, and 2-deoxy-D-ribose. In some embodiments, R⁵ is selected from the group consisting of glucose, galactose, deoxyglucose, fructose, ribose, and deoxyribose. In some embodiments, R⁵ is a deoxyglucose. In some embodiment, R⁵ is

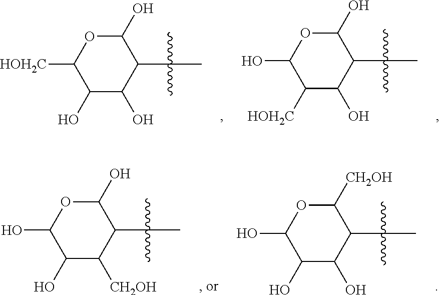 [SMILES: *C1C(O)OC(CO)C(O)C1O ; *C1C(O)OC(O)C(CO)C1O ; *C1C(O)OC(O)C(O)C1CO ; *C1C(CO)OC(O)C(O)C1O]

In some embodiments, R⁵ is

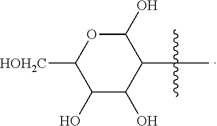 [SMILES: *C1C(O)OC(CO)C(O)C1O]

In some embodiments, one, two, or three H atoms in —(CH₂)ₙ— are each independently replaced with halogen, —OH, —COOH, —COO(C₁₋₄ alkyl), optionally substituted C₁₋₃₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, and optionally substituted 5-10 membered heteroaryl.

In some embodiments, n is an integer between 0 and 3. In some embodiments, n is 0, 1, or 2. In some embodiments, n is 0. In some embodiments, n is 1. In some embodiments, n is 2.

In some embodiments, R⁴ is a L-amino acid residue. In some embodiments, R⁴ is a L-Lys or L-Glu residue. In some embodiments, R⁴ is an amino acid residue selected from Gly, Ala, Phe, Tyr, Glu, Leu, Ser, Arg, Gln, Val, Lys, Thr, Asn, Met, Cys, Trp, Asp, His, Pro, or Ile.

In some embodiments, R⁵ is H, methyl, phenyl,

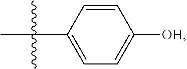 [SMILES: *c1ccc(O)cc1]

—COOH, —CH(CH₃)₂, —OH, —OCH₃, —NHC(═NH)NH₂, —CONH₂, —CH(CH₃)₂, —NH₂, —SH, —S-CH₃—CH(CH₃)(CH₂OH), indolyl, —CH(CH₃)(CH₂CH₃), imidazolyl, or pyrrolidinyl. In some embodiments, R⁵ is H. In some embodiments, R⁵ is methyl. In some embodiments, R⁵ is phenyl. In some embodiments, R⁵ is

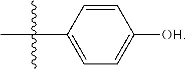 [SMILES: *c1ccc(O)cc1]

In some embodiments, R⁵ is —COOH. In some embodiments, R⁵ is —CH(CH₃)₂. In some embodiments, R⁵ is —OH. In some embodiments, R⁵ is —NHC(═NH)NH₂. In some embodiments, R⁵ is —CONH₂. In some embodiments, R⁵ is —CH(CH₃)₂. In some embodiments, R⁵ is —NH₂. In some embodiments, R⁵ is SH. In some embodiments, R⁵ is S-CH₃. In some embodiments, R⁵ is —CH(CH₃)(CH₂OH). In some embodiments, R⁵ is indolyl. In some embodiments, R⁵ is CH(CH₃)(CH₂CH₃). In some embodiments, R⁵ is imidazolyl. In some embodiments, R⁵ is pyrrolidinyl.

In some embodiments, R³ is acyl, D-glucose, 2-deoxy-D-glucose, D-ribose, or 2-deoxy-D-ribose. In some embodiments, R³ is

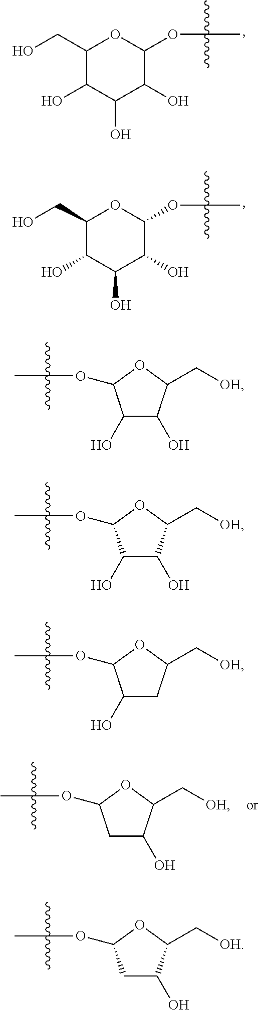

In some embodiments, R³ is

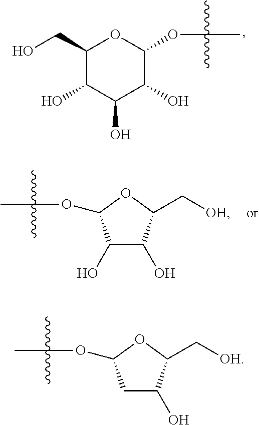

R³ is H or C₁₋₆ alkyl. In some embodiments, R³ is H.

In some embodiments, the compound described herein is selected from

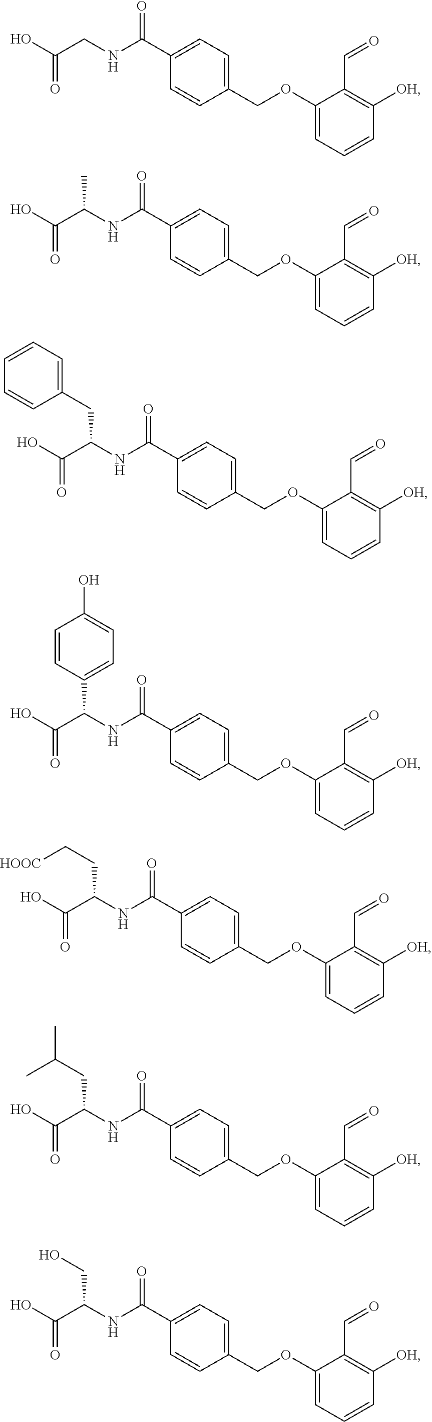

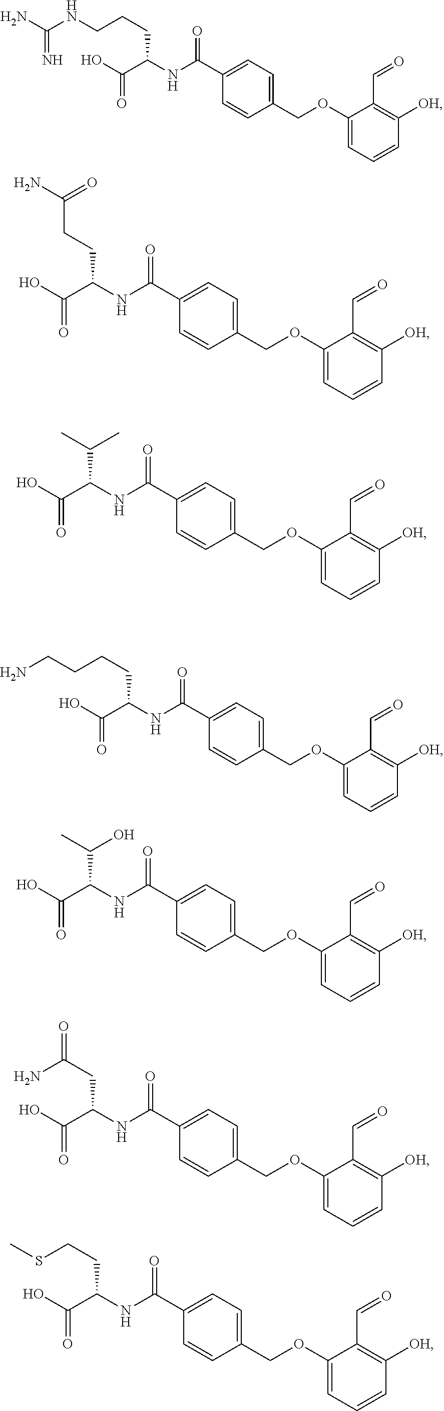

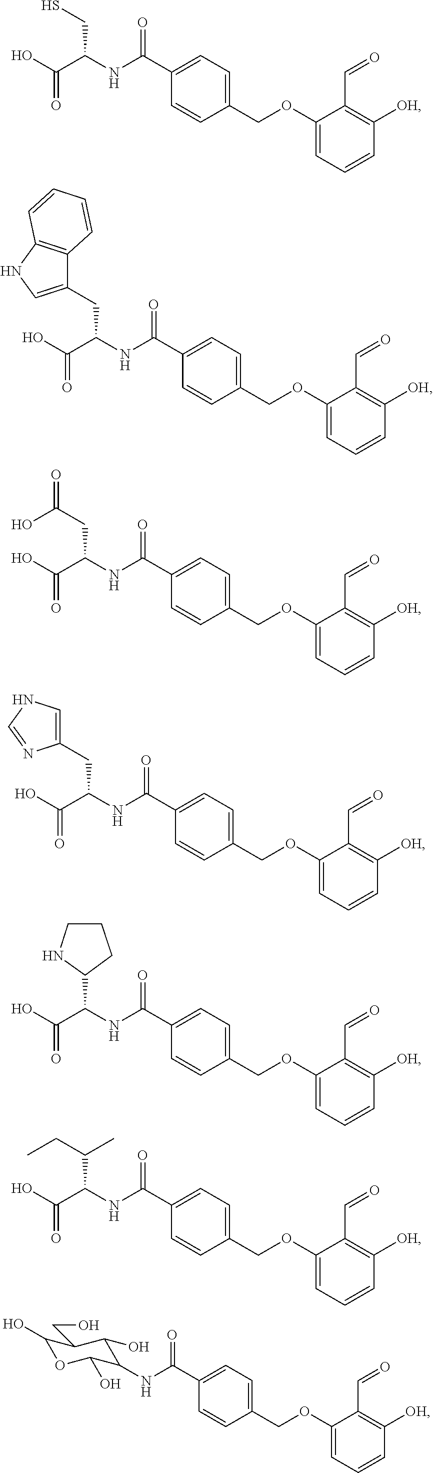

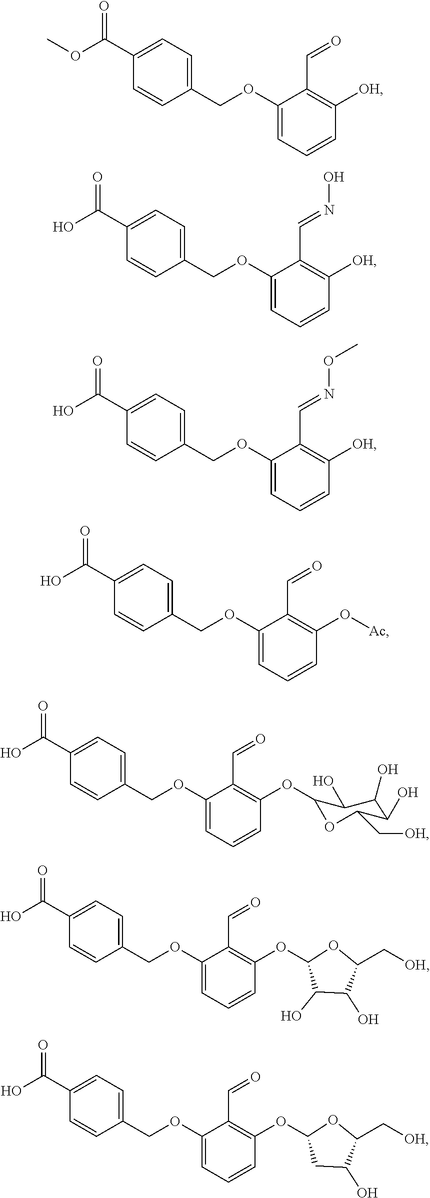

or pharmaceutically acceptable salts thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Administration and Pharmaceutical Compositions

Some embodiments relate to a pharmaceutical composition comprising a therapeutically effective amount of a compound or a pharmaceutically acceptable salt thereof described herein and a pharmaceutically acceptable excipient.

In some embodiments, the composition described herein further comprises one or more additional agents.

In some embodiments, the composition described herein further comprises one or more immune checkpoint inhibitors. In some embodiments, each of the one or more immune checkpoint inhibitors is independently an inhibitor of PD-1, PD-L1, PD-L2, PD-L3, PD-L4, CTLA-4, LAG3, B7-H3, B7-H4, KIR, TIM3, CEACAM, VISTA, BTLA, TIGIT, LAIR1, CD160, 2B4 or TGFR beta. In some embodiments, each of the one or more immune checkpoint inhibitors is independently an inhibitor of PD-1, PD-L1, PD-L2, PD-L3, PD-L4, CTLA-4, LAG3, B7-H3, B7-H4, KIR or TIM3. In some embodiments, the immune checkpoint inhibitor is a PD-1 inhibitor. In some embodiments, the immune checkpoint inhibitor is a binding ligand of PD-L 1 . In some embodiments, the immune checkpoint inhibitor is a PD-L1 inhibitor. In some embodiments, the immune checkpoint inhibitor is a PD-L2 inhibitor or a combined PD-L1/PD-L2 inhibitor. In some embodiments, the immune checkpoint inhibitor is a CTLA-4 inhibitor.

In some embodiments, the one or more immune checkpoint inhibitor is an inhibitor of PD-1, e.g., human PD-1. In another embodiment, the one or more immune checkpoint inhibitor is an inhibitor of PD-L1, e.g., human PD-L1 . In one embodiment, the inhibitor of PD-1 or PD-L1 is an antibody molecule to PD-1 or PD-L1. The PD-1 or PD-L1inhibitor can be administered alone, or in combination with other one or more immune checkpoint inhibitor, e.g., in combination with an inhibitor of LAG-3, TIM-3, CEACAM (e.g., CEACAM-1, -3 and/or -5) or CTLA-4. In an exemplary embodiment, the inhibitor of PD-1 or PD-L1, e.g., the anti-PD-1 or PD-Ll antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In another embodiment, the inhibitor of PD-1 or PD-L1, e.g., the anti-PD-1 or PD-Ll antibody molecule, is administered in combination with a CEACAM inhibitor (e.g., CEACAM-1, -3 and/or -5 inhibitor), e.g., an anti-CEACAM antibody molecule. In another embodiment, the inhibitor of PD-1 or PD-L1, e.g., the anti-PD-1 or PD-L1 antibody molecule, is administered in combination with a CEACAM-1 inhibitor, e.g., an anti-CEACAM-1 antibody molecule. In another embodiment, the inhibitor of PD-1 or PD-L1, e.g., the anti-PD-1 or PD-L1 antibody molecule, is administered in combination with a CEACAM-5 inhibitor, e.g., an anti-CEACAM-5 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule. Other combinations of one or more immune checkpoint inhibitor with a PD-1 inhibitor (e.g., one or more of PD-L2, CTLA-4, TIM-3, LAG-3, CEACAM (e.g., CEACAM-1, -3 and/or -5), VISTA, BTLA, TIGIT, LAIR1, CD160, 2B4 and/or TGFR) are also within the present disclosure. Any of the antibody molecules known in the art or disclosed herein can be used in the aforesaid combinations of inhibitors of checkpoint molecule.

A variety of antibodies (Abs) can be used in the composition described herein, including antibodies having high-affinity binding to PD-1 PD-L1, PD-L2, PD-L3, or PD-L4. Human mAbs (HuMAbs) that bind specifically to PD-1 (e.g., bind to human PD-1 and may cross-react with PD-1 from other species, such as cynomolgus monkey) with high affinity have been disclosed in U.S. Pat. No. 8,008,449, which is incorporated herein by reference in its entirety. HuMAbs that bind specifically to PD-L1 with high affinity have been disclosed in U.S. Pat. No. 7,943,743, which is incorporated herein by reference in its entirety. Other anti-PD-1 mAbs have been described in, for example, U.S. Pat. Nos. 6,808,710, 7,488,802 and 8,168,757, and PCT Publication No. WO 2012/145493, all of which are incorporated herein by reference in their entireties. Anti-PD-L1 mAbs have been described in, for example, U.S. Pat. Nos. 7,635,757, 8,217,149, 10,723,799, U.S. Publication No. 2009/0317368, and PCT Publication Nos. WO 2011/066389 and WO 2012/14549, all of which are incorporated herein by reference in their entireties.

In some embodiments, the anti-PD-1 HuMAbs can be selected from 17D8, 2D3, 4H1, 5C4 (also referred to herein as nivolumab), 4A1 1, 7D3 and 5F4, all of which are described in U.S. Pat. No. 8,008,449. In some embodiments, the anti-PD-1 HuMAbs can be selected from 3G10, 12A4 (also referred to herein as BMS-936559), 10A5, 5F8, 10H10, 1B12, 7H1, 1 1E6, 12B7, and 13G4, all of which are described in U.S. Pat. No. 7,943,743.

In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and W02006/121168, which are incorporated herein by reference in their entirety.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, WO2009/114335, and U.S. Pat. No. 8,354,509, all of which are incorporated herein by reference in their entirety.

In some embodiments, the anti-PD-1 antibody is Pidilizumab. Pidilizumab (CT-011; Cure Tech) is a humanized IgGlk monoclonal antibody that binds to PD1. Pidilizumab and other humanized anti-PD-1 monoclonal antibodies are disclosed in WO2009/101611. Other anti-PD1 antibodies are disclosed in U.S. Pat. No. 8,609,089, US 2010028330, and/or US 20120114649, all of which are incorporated herein by reference in their entirety. Other anti-PD1 antibodies include AMP 514 (Amplimmune).

In some embodiments, the PD-1 inhibitor is an immunoadhesin (e.g., an immunoadhesin comprising an extracellular or PD-1 binding portion of PD-Ll or PD-L2 fused to a constant region (e.g., an Fc region of an immunoglobulin sequence). In some embodiments, the PD-1 inhibitor is AMP-224.

In one embodiment, the PD-1 inhibitor is MDX-1105. MDX-1105, also known as BMS-936559, is an anti-PD-1 antibody described in WO2007/005874, which is incorporated herein by reference in its entirety.

In one embodiment, the PD-1 inhibitor is YW243.55.S70. The YW243.55.S70 antibody is an anti-PD-1 described in WO 2010/077634 (heavy and light chain variable region sequences shown in SEQ ID Nos. 20 and 21, respectively), which is incorporated herein by reference in its entirety.

In one embodiment, the PD-1 inhibitor is MDPL3280A (Genentech/Roche). MDPL3280A is a human Fc optimized IgG1 monoclonal antibody that binds to PD-1 . MDPL3280A and other human monoclonal antibodies to PD-1 are disclosed in U.S. Pat. No. 7,943,743 and U.S Publication No.: 20120039906, both of which are incorporated herein by reference in their entirety.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in WO2010/027827 and WO2011/066342, both of which are incorporated herein by reference in their entirety).

In one embodiment, the LAG-3 inhibitor is an anti-LAG-3 antibody molecule. In one embodiment, the LAG-3 inhibitor is BMS-986016.

In some embodiments, the CTLA-4 inhibitor is an inhibitor that binds between between the B7.1 (CD80) and B7.2 (CD86) ligands of the APC and the CD28 and CTLA-4 receptors of the CD4+T-lymphocyte (Sharpe, A. H. et al. (2002) Nature Rev. Immunol. 2:116-126; Lindley, P. S. et al. (2009) Immunol. Rev. 229:307-321; both of which are incorporated herein by reference in their entirety). Binding of B7.1 or B7.2 to CD28 stimulates T cell activation, whereas binding of B7.1 or B7.2 to CTLA-4 inhibits such activation (Dong, C. et al. (2003) Immunolog. Res. 28(1):39-48; Greenwald, R. J. et al. (2005) Ann. Rev. Immunol. 23:515-548; both of which are incorporated herein by reference in their entirety). CD28 is constitutively expressed on the surface of T cells (Gross, J., et al. (1992) J. Immunol. 149:380-388, which is incorporated herein by reference in its entirety), whereas CTLA4 expression is rapidly up-regulated following T-cell activation (Linsley, P. et al. (1996) Immunity 4:535-543, which is incorporated herein by reference in its entirety). Exemplary anti-CTLA4 antibodies that can be used in the methods disclosed herein include, but are not limited to, Tremelimumab (IgG2 monoclonal antibody available from Pfizer, formerly known as ticilimumab, CP-675,206); and Ipilimumab (CTLA-4 antibody, also known as MDX-010, CAS No. 477202-00-9). Antibodies to T cell costimulatory molecules such as CTLA-4 (e.g., U.S. Pat. No. 5,811,097, which is incorporated herein by reference in its entirety), CTLA-4 inhibitor (e.g., CP-675,206, ipilimumab).

In another embodiment, the inhibitor of CEACAM (e.g., CEACAM-1, -3 and/or -5) is an anti-CEACAM antibody molecule. In one embodiment, the inhibitor of CEACAM is an anti-CEACAM-1 antibody as described in WO 2010/125571, WO 2013/82366 and WO 2014/022332 (all of which are incorporated herein by reference in their entirety), e.g., a monoclonal antibody 34B1, 26H7, and 5F4 or a recombinant form thereof, as described in, e.g., US 2004/0047858, U.S. Pat. No. 7,132,255 and WO 99/52552 (all of which are incorporated herein by reference in their entirety). In other embodiments, the anti-CEACAM antibody is an anti-CEACAM-1 and/or anti-CEACAM-5 antibody molecule as described in, e.g., WO 2010/125571, WO 2013/054331 and US 2014/0271618 (all of which are incorporated herein by reference in their entirety).

In some embodiments, the one or more immune checkpoint inhibitor is an antibody selected from α-CD3-APC, α-CD3-APC-H7, α-CD4-ECD, α-CD4-PB, α-CD8-PE-Cy7, α-CD-8-PerCP-Cy5.5, α-CD 1 lc-APC, α-CD11b-PE-Cy7, α-CD11b-AF700, α-CD14-FITC, α-CD16-PB, α-CD19-AF780, α-CD19-AF700, α-CD2O-PO, α-CD25-PE-Cy7, α-CD40-APC, α-CD45-Biotin, Streptavidin-BV605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-PE, a-CTLA4-PE-Cy7, α-FoxP3-AF488 (clone 259D), IgGl-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, and CD137 as disclosed in U.S. Publication No. 2018/0029531, which is incorporated herein by reference in its entirety.

In some embodiments, the composition described herein further comprises plinabulin.

In some embodiments, the composition described herein further comprises one or more additional chemotherapeutic agent.

In some embodiments, the composition described herein further comprises one or more pharmaceutically acceptable excipients.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound may be determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parenteral routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan. In some embodiments, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day.

### Method of Treatment

Some embodiments relate to a method of enhancing immune response in a subject, comprising administering the compound or composition described herein.

Some embodiments relate to a method of treating cancer, comprising administering the compound or composition described herein to a subject in need thereof.

Some embodiments relate to a method of enhancing immune response in a cancer patient, comprising administering the compound or composition described herein.

Some embodiments relate to methods for stimulating an immune response, enhancing immunogenicity of an immunogen, and methods of treating an infection, an autoimmune disease, an allergy, using the compound or composition described herein.

Some embodiments relate to methods of providing co-stimulation of T-cell activation against cancer by co-administering a compound of formula (I), one or more immune checkpoint inhibitor. Some embodiments relate to methods of providing co-stimulation of natural killer cells against cancer by co-administering a compound of formula (I), one or more immune checkpoint inhibitor.

In some embodiments, the patient is administered a vaccine or immunization.

In some embodiments, the methods described herein comprises administering one or more additional agents.

In some embodiments, the methods described herein comprises administering one or more checkpoint inhibitors.

In some embodiments, the immune checkpoint inhibitor is an inhibitor of PD-1, PD-L1, PD-L2, PD-L3, PD-L4, CTLA-4, LAG3, B7-H3, B7-H4, KIR or TIM3. In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a PD-L2 inhibitor or a combined PD-L1PD-L2 inhibitor. In some embodiments, the immune checkpoint inhibitor is a CTLA-4 inhibitor. In some embodiments, the cancer comprises cancer cells expressing a binding ligand of PD-1. In some embodiments, the binding ligand of PD-1 is PD-Ll. In some embodiments, the binding ligand of PD-1 is PD-L2.

In some embodiments, the method of treating cancer described herein further includes identifying cancer cells expressing a binding ligand of PD-1. In some embodiments, the method of treating cancer described herein further includes identifying cancer cells expressing PD-L1. In some embodiments, the method of treating cancer described herein further includes identifying cancer cells expressing PD-L2. In some embodiments, the method of treating cancer described herein further includes identifying cancer cells expressing PD-L3 or PD-L4.

In some embodiments, identifying cancer cells expressing a binding ligand of PD-1 includes using an assay to detect the presence of the binding ligand. Examples of applicable assay include but are not limited to PD-L1 IHC 22C3 pharmDx kit and PD-L1IHC 28-8 pharmDx available from Dako.

In some embodiments, the cancer comprises cancer calls expressing a binding ligand of CTLA-4. In some embodiments, the binding ligand of CTLA-4 is B7.1 or B7.2.

In some embodiments, the method of treating cancer described herein further includes identifying cancer cells expressing a binding ligand of CTLA-4. In some embodiments, the method of treating cancer described herein further includes identifying cancer cells expressing B7.1 or B7.2.

In some embodiments, the immune checkpoint inhibitor is nivolumab, pembrolizumab, pidilizumab, ipilimumab, dacarbazine, BMS 936559, atezolizumab, durvalumab, cemiplimab, avelumab, spartalizumab, camrelizumab, sintilimab, tislelizumab, toripalimab, dostarlimab, INCMGA00012, AMP-224, AMP-514, KN035, AUNP12, CA-170, BMS 986189, PD1.1 (CNCM deposit number 1-4080), PD1.2 (CNCM deposit number I-4081), PD1.3 (CNCM deposit number I-4122), or any combinations thereof.

In some embodiments, cancer is head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloma, lymphoma, or leukemia. In some embodiments, the cancer is renal cell carcinoma, malignant melanoma, non-small cell lung cancer (NSCLC), ovarian cancer, Hodgkin's lymphoma or squamous cell carcinoma. In some embodiments, the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma. In some embodiments, the cancer is a solid tumor or hematological cancer.

In some embodiments, the cancer does not have any cells expressing PD-1, PD-L1, or PD-L2 at detectable levels.

In some embodiments, the combination of a compound of Formula (I) and PD-1 inhibitor (or PD-L1 inhibitor/PD-L2 inhibitor) exhibits better safety profile and lower toxicity than the combination of CTLA-4 and PD-1 inhibitor (or PD-L1 inhibitor/PD-L2 inhibitor). In some embodiments, the therapeutic index for the combination of a compound of Formula (I) and PD-1 inhibitor (or PD-L1 inhibitor/PD-L2 inhibitor) is greater than the therapeutic index of the combination of CTLA-4 and PD-1 inhibitor (or PD-L1 inhibitor/PD-L2 inhibitor).

In some embodiments, the cancer is the cancer is selected from the group consisting of a melanoma, a pancreatic cancer, a colorectal adenocarcinoma, a brain tumor, acute lymphoblastic leukemia, chronic lymphocytic leukemia, hormone refractory metastatic prostate cancer, metastatic breast cancer, non-small cell lung cancer, renal cell carcinoma, head and neck cancer, prostate cancer, colon cancer, anaplastic thyroid cancer.

Some embodiments include co-administering a composition, and/or pharmaceutical composition described herein, with an additional medicament. For example, as described above, some embodiments include co-administering a compound of Formula (I) described herein with one or more immune checkpoint inhibitor, some embodiments include co-administering a compound of Formula (I) described herein with one or more immune checkpoint inhibitor and plinabulin, and some embodiments include co-administering a compound of Formula (I) described herein with plinabulin. By “co-administration,” it is meant that the two or more agents are administered in such a manner that administration of one or more agent has an effect on the efficacy and/or safety of the one or more other agent, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally or intravenously. In another embodiment, the agents are administered through different routes, such as one being administered orally and another being administered i.v. In some embodiments, the time period between administration of one or more agent and administration of the co-administered one or more agent can be about 5min, 10 min, 20 min, 30min, 40min, 50min, 1 hour, 2 hours, 3 hours, 5 hours, 8 hours, 10 hours, 12 hours, 15 hours, 18 hours, 20 hours, 24 hours, 36 hours, 48 hours, 3 days, 4 days, 5 days, 6 days, 7 days, 10 days, 14 days, 21 days, 28 days, or 30 days. In some embodiments, the time period between administration of one or more agent and administration of the co-administered one or more agent can be in the range of about 1 min-5 min, 1 min-10 min, 1 min-20 min, 1 min-30 min, 1 min-40 min, 1 min-50 min, 1 min-1 h, 1 min-2 h, 1 min-4h, 1 min-6 h, 1 min-8 h, 1 min-10 h, 1 min-12 h, 1 min-24 h, 1 min-36 h, 1 min-48 h, 1 min-60 h, 1 min-72 h, 5 min-10 min, 5 min-20 min, 5 min-30 min, 5 min-40 min, 5 min-50 min, 5 min-1 h, 5 min-2 h, 5 min-4 h, 5 min-6 h, 5 min-8 h, 5 min-10 h, 5 min-12 h, 5 min-24 h, 5 min-36 h, 5 min-48 h, 5 min-60 h, 5 min-72 h, 10 min-20 min, 10 min-30 min, 10 min-40 min, 10 min-50 min, 10 min-1 h, 10 min-2 h, 10 min-4 h, 10 min-6 h, 10 min-8 h, 10 min-10 h, 10 min-12 h, 10 min-24 h, 10 min-36 h, 10 min-48 h, 10 min-60 h, 10 min-72 h, 30 min-40 min, 30 min-50 min, 30 min-1 h, 30 min-2 h, 30 min-4 h, 30 min-6 h, 30 min-8 h, 30 min-10 h, 30 min-12 h, 30 min-24 h, 30 min-36 h, 30 min-48 h, 30 min-60 h, 30 min-72 h, 1 h-2 h, 1 h-4 h, 1 h-6 h, 1 h-8 h, 1 h-10 h, 1 h-12 h, 1 h-24 h, 1 h-36 h, 1 h-48 h, 1 h-60 h, 1 h-72 h, 6 h-8 h, 6 h-10 h, 6 h-12 h, 6 h-24 h, 6 h-36 h, 6 h-48 h, 6 h-60 h, 6 h-72 h, 12 h-24 h, 12 h-36 h, 12 h-48 h, 12 h-60 h, or 12 h-72 h.

The actual dose of the active compounds described herein depends on the specific compound, and on the condition to be treated; the selection of the appropriate dose is well within the knowledge of the skilled artisan. In some embodiments, the method described herein comprises administering the compound of Formula (I) at a dose in the range of from about 0.01 mg/kg to about 250 mg/kg of body weight, from about 0.1 mg/kg to about 200 mg/kg of body weight, from about 0.25 mg/kg to about 120 mg/kg of body weight, from about 0.5 mg/kg to about 70 mg/kg of body weight, from about 1.0 mg/kg to about 50 mg/kg of body weight, from about 1.0 mg/kg to about 15 mg/kg of body weight, from about 2.0 mg/kg to about 15 mg/kg of body weight, from about 3.0 mg/kg to about 12 mg/kg of body weight, or from about 5.0 mg/kg to about 10 mg/kg of body weight. In some embodiments, the method described herein comprises administering the compound of Formula (I) at a dose in the range of 0.5-1, 0.5-2, 0.5-3, 0.5-4, 0.5-5, 0.5-6, 0.5-7, 0.5-8, 0.5-9, 0.5-10, 1-2, 1-3, 1-4, 1-5, 1-6, 1-7, 1-8, 1-9, 1-10, 1-20, 1-30, 1-40, 1-50, 1-60, 1-70, 1-80, 1-90, 1-100, 2.5-5, 2.5-10, 2.5-20, 2.5-30, 2.5-40, 2.5-50, 2.5-60, 2.5-70, 2.5-80, 2.5-90, 2.5-100, 3-5, 3-10, 3-20, 3-30, 3-40, 3-50, 3-60, 3-70, 3-80, 3-90, 3-100, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 80-100, 80-150, 80-200, 80-250, 80-300, 90-100, 90-150, 90-200, 90-250, 90-300, 90-350, 90-400, 100-150, 100-200, 100-250, 100-300, 100-350, or 100-400 mg/kg of body weight. In some embodiments, the compound of Formula (I) described herein may be administered at a dose of about 0.1, 0.25, 0.5, 1, 2, 3, 4, 5, 6, 7, 8, 9, 10, 11, 12, 13, 14, 15 ,16, 17, 18,19, 20, 22.5, 25, 27.5, 30, 35, 40, 45, 50, 55, 60, 65, 70, 75, 80, 85, 90, 95, or 100 mg/kg of body weight.

In some embodiments, the method described herein comprises administering the compound of formula (I) at a dose of about 3 mg/kg. In some embodiments, the method described herein comprises administering the compound of formula (I) at a dose of about 3 mg/kg every three weeks for a total of four doses.

In some embodiments, the compound of Formula (I) is administered at an amount of about 0.5-1, 0.5-2, 0.5-3, 0.5-4, 0.5-5, 0.5-6, 0.5-7, 0.5-8, 0.5-9, 0.5-10, .2.5-3, 2.5-4, 2.5-5, 2.5-6, 2.5-7, 2.5-8, 2.5-9, 2.5-10,3-10, 5-10, 1-10, 1-20, 1-30, 1-40, 1-50, 1-60, 1-70, 1-80, 1-90, 1-100, 2.5-10, 2.5-20, 2.5-30, 2.5-40, 2.5-50, 2.5-60, 2.5-70, 2.5-80, 2.5-90, 2.5-100, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80,20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 70-500, 70-750, 70-1000, 70-1500, 70-2000, 70-3000, 80-100, 80-150, 80-200, 80-250, 80-300, 80-500, 80-750, 80-1000, 80-1500, 80-2000, 80-3000, 90-100, 90-150, 90-200, 90-250, 90-300, 90-350, 90-400, 90-500, 90-750, 90-1000, 90-1500, 90-2000, 90-3000, 100-150, 100-200, 100-250, 100-300, 100-350, 100-400, 100-500, 100-600, 100-700, 100-800, 100-900, 100-1000, 100-1500, 100-2000, 100-2500, 100-3000, 100-3500, 100-4000, 200-500, 200-700, 200-1000, 200-1500, 200-2000, 200-2500, 200-3000, 200-3500, 200-4000, 500-1000, 500-1500, 500-2000, 500-2500, 500-3000, 500-3500, or 500-4000 mg per dose. In some embodiments, the compound of Formula (I) is administered at an amount of about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10, 12.5, 15, 17.5, 20, 22.5, 25, 27.5, 30, 40, 50, 60, 70, 80, 90, 100, 150, 200, 250, 300, 350,400, 450, 500, 550, 600, 650, 700, 750, 800, 850, 900, 1000, 1100, 1200, 1250, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg per dose. In some embodiments, the compound of formula (I) is administered at an amount of about 25 mg, 50 mg, or 100 mg per dose.

In some embodiments, the method described herein comprises administering one or more check point inhibitors at a dose in the range of about 0.5-1, 0.5-2, 0.5-3, 0.5-4, 0.5-5, 0.5-6, 0.5-7, 0.5-8, 0.5-9, 0.5-10, 1-2, 1-3, 1-4, 1-5, 1-6, 1-7, 1-8, 1-9, 1-10, 1-20, 1-30, 1-40, 1-50, 1-60, 1-70, 1-80, 1-90, 1-100, 2-3, 2-4, 2-5, 2-6, 2-7, 2-8, 2-9, 2-10, 2-20, 2-30, 2-40, 2-50, 2-60, 2-70, 2-80, 2-90, 2-100, 2.5-3, 2.5-3.5, 2.5-4, 2.5-5, 2.5-6, 2.5-7, 2.5-9, 2.5-10, 3-4, 3-5, 3-6, 3-7, 3-8, 3-9, 3-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 80-100, 80-150, 80-200, 80-250, 80-300, 90-100, 90-150, 90-200, 90-250, 90-300, 90-350, 90-400, 100-150, 100-200, 100-250, 100-300, 100-350, or 100-400 mg/kg of the body weight. In some embodiments, the method described herein comprised administering one or more checkpoint inhibitors at a dose of about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10, 12.5, 15, 17.5, 20, 22.5, 25, 27.5, 30, 40, 50, 60, 70, 80, 90 or 100 mg/kg of the body weight.

In some embodiments, the one or more check point inhibitor are administered at an amount of about 0.5-1, 0.5-2, 0.5-3, 0.5-4, 0.5-5, 0.5-6, 0.5-7, 0.5-8, 0.5-9, 0.5-10, 1-10, 1-20, 1-30, 1-40, 1-50, 1-60, 1-70, 1-80, 1-90, 1-100, 1-150, 1-200, 1-250, 1-300, 1-500, 2.5-3, 2.5-4, 2.5-5, 2.5-6, 2.5-7, 2.5-8, 2.5-9, 2.5-10, 2.5-20, 2.5-30, 2.5-40, 2.5-50, 2.5-60, 2.5-70, 2.5-80, 2.5-90, 2.5-100, 2.5-200, 2.5-250, 2.5-300, 2.5-500, 3-10, 3-20, 3-30, 3-40, 3-50, 3-60, 3-70, 3-80, 3-90, 3-100, 3-200, 3-250, 3-300, 3-500, 5-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 70-500, 70-750, 70-1000, 70-1500, 70-2000, 70-3000, 80-100, 80-150, 80-200, 80-250, 80-300, 80-500, 80-750, 80-1000, 80-1500, 80-2000, 80-3000, 90-100, 90-150, 90-200, 90-250, 90-300, 90-350, 90-400, 90-500, 90-750, 90-1000, 90-1500, 90-2000, 90-3000, 100-150, 100-200, 100-250, 100-300, 100-350, 100-400, 100-500, 100-600, 100-700, 100-800, 100-900, 100-1000, 100-1500, 100-2000, 100-2500, 100-3000, 100-3500, 100-4000, 200-500, 200-700, 200-1000, 200-1500, 200-2000, 200-2500, 200-3000, 200-3500, 200-4000, 500-1000, 500-1500, 500-2000, 500-2500, 500-3000, 500-3500, or 500-4000 mg per dose. In some embodiments, the one or more check point inhibitors are administered at an amount of about 1, 2, 3, 4, 5, 6, 7, 8, 9, 10, 12, 14, 16, 18, 20, 22, 25, 27, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900, 1000, 1100, 1200, 1300, 1400, 1500, 1600, 1700, 1800, 1900, or 2000 mg per dose.

In some embodiments, the method described herein comprises administering an inhibitor of PD-1 at a dose in the range of about 0.5-1, 0.5-2, 0.5-3, 0.5-4, 0.5-5, 0.5-6, 0.5-7, 0.5-8, 0.5-9, 0.5-10, 1-2, 1-3, 1-4, 1-5, 1-6, 1-7, 1-8, 1-9, 1-10, 1-20, 1- 30, 1-40, 1-50, 1-60, 1-70, 1-80, 1-90, 1-100, 2-3, 2-4, 2-5, 2-6, 2-7, 2-8, 2-9, 2-10, 2-20, 2-30, 2-40, 2-50, 2-60, 2-70, 2-80, 2-90, 2-100, 2.5-3, 2.5-3.5, 2.5-4, 2.5-5, 2.5-6, 2.5-7, 2.5-9, 2.5-10, 3-4, 3-5, 3-6, 3-7, 3-8, 3-9, 3-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 80-100, 80-150, 80-200, 80-250, 80-300, 90-100, 90-150, 90-200, 90-250, 90-300, 90-350, 90-400, 100-150, 100-200, 100-250, 100-300, 100-350, or 100-400 mg/kg of the body weight. In some embodiments, the method described herein comprises administering the inhibitor of PD-1 at a dose of about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10, 12.5, 15, 17.5, 20, 22.5, 25, 27.5, 30, 40, 50, 60, 70, 80, 90 or 100 mg/kg of the body weight. In some embodiments, the inhibitor of PD-1 is administered at a dose of about 3 mg/kg. In some embodiments, the inhibitor of PD-1 is administered at a dose of about 2 mg/kg.

In some embodiments, the PD-1 inhibitor is administered at an amount of about 1-10, 1-20, 1-30, 1-40, 1-50, 1-60, 1-70, 1-80, 1-90, 1-100, 1-150, 1-200, 1-250, 1-300, 1-500, 2.5-3, 2.5-4, 2.5-5, 2.5-6, 2.5-7, 2.5-8, 2.5-9, 2.5-10, 2.5-20, 2.5-30, 2.5-40, 2.5-50, 2.5-60, 2.5-70, 2.5-80, 2.5-90, 2.5-100, 2.5-200, 2.5-250, 2.5-300, 2.5-500, 3-10, 3-20, 3-30, 3-40, 3-50, 3-60, 3-70, 3-80, 3-90, 3-100, 3-200, 3-250, 3-300, 3-500, 5-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 70-500, 70-750, 70-1000, 70-1500, 70-2000, 70-3000, 80-100, 80-150, 80-200, 80-250, 80-300, 80-500, 80-750, 80-1000, 80-1500, 80-2000, 80-3000, 90-100, 90-150, 90-200, 90-250, 90-300, 90-350, 90-400, 90-500, 90-750, 90-1000, 90-1500, 90-2000, 90-3000, 100-150, 100-200, 100-250, 100-300, 100-350, 100-400, 100-500, 100-600, 100-700, 100-800, 100-900, 100-1000, 100-1500, 100-2000, 100-2500, 100-3000, 100-3500, 100-4000, 200-500, 200-700, 200-1000, 200-1500, 200-2000, 200-2500, 200-3000, 200-3500, 200-4000, 500-1000, 500-1500, 500-2000, 500-2500, 500-3000, 500-3500, or 500-4000 mg per dose. In some embodiments, the PD-1 inhibitor is administered at an amount of about 10-30, 10-50, 10-80, 10-100, 10-125, 10-150, 10-175, 10-200, 10-250, 10-300, 10-400, 20-50, 20-100, 20-125, 20-150, 20-175, 20-200, 20-250, 20-300, 20-400, 30-50, 30-80, 30-100, 30-125, 30-150, 30-175, 30-200, 30-250, 30-300, 30-400, 40-50, 40-80, 40-100, 40-125, 40-150, 40-175, 40-200, 40-250, 40-300, 40-400, 50-80, 50-100, 50-125, 50-150, 50-175, 50-200, 50-250, 50-300, or 50-400 mg per dose.

In some embodiments, the method described herein comprises administering an inhibitor of PD-L1 at a dose in the range of about 0.5-1, 0.5-2, 0.5-3, 0.5-4, 0.5-5, 0.5-6, 0.5-7, 0.5-8, 0.5-9, 0.5-10, 1-2, 1-3, 1-4, 1-5, 1-6, 1-7, 1-8, 1-9, 1-10, 1-20, 1- 30, 1-40, 1-50, 1-60, 1-70, 1-80, 1-90, 1-100, 2-3, 2-4, 2-5, 2-6, 2-7, 2-8, 2-9, 2-10, 2-20, 2-30, 2-40, 2-50, 2-60, 2-70, 2-80, 2-90, 2-100, 2.5-3, 2.5-3.5, 2.5-4, 2.5-5, 2.5-6, 2.5-7, 2.5-9, 2.5-10, 3-4, 3-5, 3-6, 3-7, 3-8, 3-9, 3-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 80-100, 80-150, 80-200, 80-250, 80-300, 90-100, 90-150, 90-200, 90-250, 90-300, 90-350, 90-400, 100-150, 100-200, 100-250, 100-300, 100-350, or 100-400 mg/kg of the body weight. In some embodiments, the method described herein comprises administering the inhibitor of PD-L1 at a dose of about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10, 12.5, 15, 17.5, 20, 22.5, 25, 27.5, 30, 40, 50, 60, 70, 80, 90 or 100 mg/kg of the body weight.

In some embodiments, the PD-L1 inhibitor (e.g., atezolizumab) is administered at an amount of about 1-10, 1-20, 1-30, 1-40, 1-50, 1-60, 1-70, 1-80, 1-90, 1-100, 1-150, 1-200, 1-250, 1-300, 1-500, 2.5-3, 2.5-4, 2.5-5, 2.5-6, 2.5-7, 2.5-8, 2.5-9, 2.5-10, 2.5-20, 2.5-30, 2.5-40, 2.5-50, 2.5-60, 2.5-70, 2.5-80, 2.5-90, 2.5-100, 2.5-200, 2.5-250, 2.5-300, 2.5-500, 3-10, 3-20, 3-30, 3-40, 3-50, 3-60, 3-70, 3-80, 3-90, 3-100, 3-200, 3-250, 3-300, 3-500, 5-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 70-500, 70-750, 70-1000, 70-1500, 70-2000, 70-3000, 80-100, 80-150, 80-200, 80-250, 80-300, 80-500, 80-750, 80-1000, 80-1500, 80-2000, 80-3000, 90-100, 90-150, 90-200, 90-250, 90-300, 90-350, 90-400, 90-500, 90-750, 90-1000, 90-1500, 90-2000, 90-3000, 100-150, 100-200, 100-250, 100-300, 100-350, 100-400, 100-500, 100-600, 100-700, 100-800, 100-900, 100-1000, 100-1500, 100-2000, 100-2500, 100-3000, 100-3500, 100-4000, 200-500, 200-700, 200-1000, 200-1500, 200-2000, 200-2500, 200-3000, 200-3500, 200-4000, 500-1000, 500-1500, 500-2000, 500-2500, 500-3000, 500-3500, or 500-4000 mg per dose. In some embodiments, the PD-L1 inhibitor is administered at an amount of about 500-1500, 600-1500, 700-1500, 800-1500, 900-1500, 1000-1500, or 1100-1300 mg per dose. In some embodiments, the PD-L1 inhibitor is administered at an amount of about 1200 mg per dose.

In some embodiments, the method described herein comprises administering the inhibitor of CTLA-4 (e.g., ipilimumab) at a dose in the range of about 0.5-1,1, 0.5-2, 0.5-3, 0.5-4, 0.5-5, 0.5-6, 0.5-7, 0.5-8, 0.5-9, 0.5-10, 1-2, 1-3, 1-4, 1-5, 1-6, 1-7, 1- 8, 1-9, 1-10, 1-20, 1-30, 1-40, 1-50, 1-60, 1-70, 1-80, 1-90, 1-100, 2-3, 2-4, 2-5, 2-6, 2-7, 2-8, 2-9, 2-10, 2-20, 2-30, 2-40, 2-50, 2-60, 2-70, 2-80, 2-90, 2-100, 2.5-3, 2.5-3.5, 2.5-4, 2.5-5, 2.5-6, 2.5-7, 2.5-9, 2.5-10, 3-4, 3-5, 3-6, 3-7, 3-8, 3-9, 3-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300 mg/kg of the body weight. In some embodiments, the method described herein comprises administering the inhibitor of CTLA-4 at a dose of about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10, 12.5, 15, 17.5, 20, 22.5, 25, 27.5, 30, 40, 50, 60, 70, 80, 90 or 100 mg/kg of the body weight. In some embodiments, the inhibitor of CTLA-4 is administered at a dose of about 3 mg/kg. In some embodiments, the inhibitor of CTLA-4 is administered at a dose of lower than 3 mg/kg. In some embodiments, the inhibitor of CTLA-4 is administered ata dose of about 0.5, 1, 1.5, 2, or 2.5 mg/kg.

In some embodiments, the CTLA-4 inhibitor is administered at an amount of about 1-10, 1-20, 1-30, 1-40, 1-50, 1-60, 1-70, 1-80, 1-90, 1-100, 1-150, 1-200, 1-250, 1-300, 1-500, 2.5-3, 2.5-4, 2.5-5, 2.5-6, 2.5-7, 2.5-8, 2.5-9, 2.5-10, 2.5-20, 2.5-30, 2.5-40, 2.5-50, 2.5-60, 2.5-70, 2.5-80, 2.5-90, 2.5-100, 2.5-200, 2.5-250, 2.5-300, 2.5-500, 3-10, 3-20, 3-30, 3-40, 3-50, 3-60, 3-70, 3-80, 3-90, 3-100, 3-200, 3-250, 3-300, 3-500, 5-10, 5-10, 5-20, 5-30, 5-40, 5-50,5-60, 5-70, 5-80, 5-90, 5-100, 7.5-10, 7.5-20, 7.5-30, 7.5-40, 7.5-50, 7.5-60, 7.5-70, 7.5-80, 7.5-90, 7.5-100, 10-10, 10-20, 10-30, 10-40, 10-50, 10-60, 10-70, 10-80, 10-90, 10-100, 10-150, 10-200, 20-30, 20-40, 20-50, 20-60, 20-70, 20-80, 20-90, 20-100, 20-150, 20-200, 30-40, 30-50, 30-60, 30-70, 30-80, 30-90, 30-100, 30-150, 30-200, 40-50, 40-60, 40-70, 40-80, 40-90, 40-100, 40-150, 40-200, 40-300, 50-60, 50-70, 50-80, 50-90, 50-100, 50-150, 50-200, 50-250, 50-300, 60-80, 60-100, 60-150, 60-200, 70-100, 70-150, 70-200, 70-250, 70-300, 70-500, 70-750, 70-1000, 70-1500, 70-2000, 70-3000, 80-100, 80-150, 80-200, 80-250, 80-300, 80-500, 80-750, 80-1000, 80-1500, 80-2000, 80-3000, 90-100, 90-150, 90-200, 90-250, 90-300, 90-350, 90-400, 90-500, 90-750, 90-1000, 90-1500, 90-2000, 90-3000, 100-150, 100-200, 100-250, 100-300, 100-350, 100-400, 100-500, 100-600, 100-700, 100-800, 100-900, 100-1000, 100-1500, 100-2000, 100-2500, 100-3000, 100-3500, 100-4000, 200-500, 200-700, 200-1000, 200-1500, 200-2000, 200-2500, 200-3000, 200-3500, 200-4000, 500-1000, 500-1500, 500-2000, 500-2500, 500-3000, 500-3500, or 500-4000 mg per dose. In some embodiments, the CTLA-4 inhibitor is administered at an amount of about 10-30, 10-50, 10-80, 10-100, 10-125, 10-150, 10-175, 10-200, 10-250, 10-300, 10-400, 20-50, 20-100, 20-125, 20-150, 20-175, 20-200, 20-250, 20-300, 20-400, 30-50, 30-80, 30-100, 30-125, 30-150, 30-175, 30-200, 30-250, 30-300, 30-400, 40-50, 40-80, 40-100, 40-125, 40-150, 40-175, 40-200, 40-250, 40-300, 40-400, 50-80, 50-100, 50-125, 50-150, 50-175, 50-200, 50-250, 50-300, or 50-400 mg per dose.

In some embodiments, the treatment schedule includes co-administration of the compound of formula (I) and one or more checkpoint inhibitors (e.g., PD-1/PD-L1 inhibitor and CTLA-4 inhibitor) once every 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors once every 2 weeks or 3 weeks. In some embodiments, the treatment schedule includes co-administration of the compound of formula (I) and one or more checkpoint inhibitors two times every 1 week, 2 weeks, 3 weeks, 4 weeks, 5 weeks, 6 weeks, 7 weeks, or 8 weeks. In some embodiments, the treatment schedule includes co-administration of a chemotherapeutic agent and plinabulin once every 1 week in a treatment cycle of 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors twice every 1 week in a treatment cycle of 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors three times every 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors four times every 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors five times every 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors six times every 1 week, 2 weeks, 3 weeks, 4 weeks, 5 weeks, 6 weeks, 7 weeks, or 8 weeks. In some embodiments, the treatment schedule includes co-administration of the compound of formula (I) and one or more checkpoint inhibitors daily every 1 week, 2 weeks, 3 weeks, or 4 weeks. In some embodiments, co-administration of the compound of formula (I) and one or more checkpoint inhibitors includes administering the compound of formula (I) prior to administering the one or more checkpoint inhibitors. In some embodiments, the treatment schedule includes co-administration of the compound of formula (I) and one or more checkpoint inhibitors 1, 2, 3, 4, 5, 6, or 7 times per day. In some embodiments, the treatment schedule includes co-administration of the compound of formula (I) and one or more checkpoint inhibitors once every 2, 3, 4, 5, or 6 days.

In some embodiments, co-administration of the compound of formula (I) and one or more checkpoint inhibitors includes administering the compound of formula (I) after administering the one or more checkpoint inhibitors. In some embodiments, co-administration of the compound of formula (I) and one or more checkpoint inhibitors includes administering the compound of formula (I) concurrently with the one or more checkpoint inhibitors. When more than one checkpoint inhibitors are administered, the two check point inhibitors can be administered separately or concurrently.

In some embodiments, when the compound of formula (I) is administered prior to the one or more checkpoint inhibitors are administered, the one or more checkpoint inhibitors can be administered about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, or 12 h after the administration of the compound of formula (I). In some embodiments, the one or more checkpoint inhibitors are administered in less than about 1 min, 5 min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of the compound of formula (I). In some embodiments, the one or more checkpoint inhibitors are administered in more than about 1 min, 5min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h after the administration of the compound of formula (I). In some embodiments, the one or more checkpoint inhibitors are administered in about 1 min-5min, 1 min-10min, 1 min-15min, 1min-20 min, 1 min-25 min, 1 min-30 min, 0.25 h-0.5 h, 0.25-0.75 h, 0.25-1 h,0.5 h-1 h, 0.5 h-2 h, 0.5 h-2.5 h, 1 h-2 h, 1 h-3 h, 1 h-5 h after the administration of the compound of formula (I). In some embodiments, the one or more checkpoint inhibitors are administered in about 1 min-5 min, 1 min-10 min, 1 min-20 min, 1 min-30 min, 1 min-40 min, 1 min-50 min, 1 min-1 h, 1 min-2 h, 1 min-4h, 1 min-6 h, 1 min-8 h, 1 min-10 h, 1 min-12 h, 1 min-24 h, 1 min-36 h, 1 min-48 h, 1 min-60 h, 1 min-72 h, 5 min-10 min, 5 min-20 min, 5 min-30 min, 5 min-40 min, 5 min-50 min, 5 min-1 h, 5 min-2 h, 5 min-4 h, 5 min-6 h, 5 min-8 h, 5 min-10 h, 5 min-12 h, 5 min-24 h, 5 min-36 h, 5 min-48 h, 5 min-60 h, 5 min-72 h, 10 min-20 min, 10 min-30 min, 10 min-40 min, 10 min-50 min, 10 min-1 h, 10 min-2 h, 10 min-4 h, 10 min-6 h, 10 min-8 h, 10 min-10 h, 10 min-12 h, 10 min-24 h, 10 min-36 h, 10 min-48 h, 10 min-60 h, 10 min-72 h, 30 min-40 min, 30 min-50 min, 30 min-1 h, 30 min-2 h, 30 min-4 h, 30 min-6 h, 30 min-8 h, 30 min-10 h, 30 min-12 h, 30 min-24 h, 30 min-36 h, 30 min-48 h, 30 min-60 h, 30 min-72 h, 1 h-2 h, 1 h-4 h, 1 h-6 h, 1 h-8 h, 1 h-10 h, 1 h-12 h, 1 h-24 h, 1 h-36 h, 1 h-48 h, 1 h-60 h, 1 h-72 h, 6 h-8 h, 6 h-10 h, 6 h-12 h, 6 h-24 h, 6 h-36 h, 6 h-48 h, 6 h-60 h, 6 h-72 h, 12 h-24 h, 12 h-36 h, 12 h-48 h, 12 h-60 h, or 12 h-72 h after the administration of the compound of formula (I).

In some embodiments, when the one or more checkpoint inhibitors are administered prior to the compound of formula (I) is administered, the one or more checkpoint inhibitors are administered about 1 min-5 min, 1 min-10 min, 1 min-15 min, 1 min-20 min, 1 min-25 min, 1 min-30 min, 0.25 h-0.5 h, 0.25-0.75 h, 0.25-1 h,0.5 h-1 h, 0.5 h-2 h, 0.5 h-2.5 h, 1 h-2 h, 1 h-3 h, or 1 h-5 h before the administration of the compound of formula (I). In some embodiments, the one or more checkpoint inhibitors are administered about 1 min, 5min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 11 h, or 12 h before the administration of the compound of formula (I). In some embodiments, the one or more checkpoint inhibitors are administered less than about 1 min, 5min, 10 min, 15 min, 20 min, 25 min, 30 min, lh, 1.5 h, 2 h, 2.5 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h before the administration of the compound of formula (I). In some embodiments, the one or more checkpoint inhibitors are administered more than about 1 min, 5min, 10 min, 15 min, 20 min, 25 min, 30 min, 1 h, 1.5 h, 2 h, 2.5 h, 3 h, 4 h, 5 h, 6 h, 7 h, 8 h, 9 h, 10 h, 12 h, 13 h, 14 h, 15 h, 16 h, 17 h, 18 h, 19 h, 20 h, 21 h, 22 h, 23 h, or 24 h before the administration of the compound of formula (I). In some embodiments, the one or more checkpoint inhibitors are administered in about 1 min-5 min, 1 min-10 min, 1 min-20 min, 1 min-30 min, 1 min-40 min, 1 min-50 min, 1 min-1 h, 1 min-2 h, 1 min-4 h, 1 min-6 h, 1 min-8 h, 1 min-10 h, 1 min-12 h, 1 min-24 h, 1 min-36 h, 1 min-48 h, 1 min-60 h, 1 min-72 h, 5 min-10 min, 5 min-20 min, 5 min-30 min, 5 min-40 min, 5 min-50 min, 5 min-1 h, 5 min-2 h, 5 min-4 h, 5 min-6 h, 5 min-8 h, 5 min-10 h, 5 min-12 h, 5 min-24 h, 5 min-36 h, 5 min-48 h, 5 min-60 h, 5 min-72 h, 10 min-20 min, 10 min-30 min, 10 min-40 min, 10 min-50 min, 10 min-1 h, 10 min-2 h, 10 min-4 h, 10 min-6 h, 10 min-8 h, 10 min-10 h, 10 min-12 h, 10 min-24 h, 10 min-36 h, 10 min-48 h, 10 min-60 h, 10 min-72 h, 30 min-40 min, 30 min-50 min, 30 min-1 h, 30 min-2 h, 30 min-4 h, 30 min-6 h, 30 min-8 h, 30 min-10 h, 30 min-12 h, 30 min-24 h, 30 min-36 h, 30 min-48 h, 30 min-60 h, 30 min-72 h, 1 h-2 h, 1 h-4 h, 1 h-6 h, 1 h-8 h, 1 h-10 h, 1 h-12 h, 1 h-24 h, 1 h-36 h, 1 h-48 h, 1 h-60 h, 1 h-72 h, 6 h-8 h, 6 h-10 h, 6 h-12 h, 6 h-24 h, 6 h-36 h, 6 h-48 h, 6 h-60 h, 6 h-72 h, 12 h-24 h, 12 h-36 h, 12 h-48 h, 12 h-60 h, or 12 h-72 h before the administration of the compound of formula (I).

The treatment cycle can be repeated as long as the regimen is clinically tolerated. In some embodiments, the treatment cycle for the compound of formula (I) and the one or more checkpoint inhibitors is repeated for n times, wherein n is an integer in the range of 2 to 30. In some embodiments, n is 2, 3, 4, 5, 6, 7, 8, 9, or 10. In some embodiments, a new treatment cycle can occur immediately after the completion of the previous treatment cycle. In some embodiments, a washout period can occur before starting a new treatment cycle. In some embodiments, the washout period can be 1 week, 2 weeks, 3 weeks, or 4 weeks. In some embodiments, the dose of the compound of formula (I) can be the same for each treatment cycle. In some embodiments, the dose of the compound of formula (I) can be different in each treatment cycle (e.g., the dose can be 20 mg for the first treatment cycle, 50 mg for the second treatment cycle, 100 mg for the third treatment cycle).

In some embodiments, after the compound of formula (I) and the one or more checkpoint inhibitors are administered in one cycle of treatment, the next treatment cycle may include administering only the compound of formula (I). In some embodiments, after the compound of formula (I) and the one or more checkpoint inhibitors are administered in one cycle of treatment, the next treatment cycle may include administering both the compound of formula (I) and the one or more checkpoint inhibitors.

In some embodiments, the compound of formula (I) is administered at a dose of about 3 mg/kg every three weeks as a treatment cycle and the treatment cycle is repeated four times. In some embodiments, the one or more checkpoint inhibitors (e.g., any one of PD-1 inhibitor, PD-L1 inhibitor, CTLA-4 inhibitor, and any combinations thereof) can be co-administered with the compound of formula (I) in each treatment cycle, In some embodiments, the one or more checkpoint inhibitors can be co-administered with the compound of formula (I) in half of the treatment cycles (e.g. the first and the third treatment cycles).

In some embodiments, the method described herein can include administering plinabulin.

In some embodiments, the method described herein can include administering a radiation therapy.

In some embodiments, the method described herein can include one or more additional medicaments. Examples of additional medicaments include other chemotherapeutic agents.

In some embodiments, the chemotherapeutic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aloxi (Palonosetron Hydrochloride), Ambochlorin (Chlorambucil), Amboclorin (Chlorambucil), Aminolevulinic Acid,\Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), B eleodaq (B elino s tat), Belinostat, B endamu s tine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), B ortezomib, B osulif (Bosutinib), Bosutinib, Brentuximab Vedotin, B usulfan, Cabazitaxel, Cabozantinib-S-Malate, CAF, Campath (Alemtuzumab), Camptosar (Irinotec an Hydrochloride), Capecitabine, CAPDX, Carac (Fluorouracil—Topical), Carboplatin, CARBOPLATIN-TAXOL, Carfilzomib, Carmubris (C armu s tine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cobimetinib, Cometriq (Cabozantinib-S-Malate), COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyv a (Obinutuzumab), Gefitinib, Gemcitabine Hydrochloride, GEMCITABINE-CISPLATIN, GEMCITABINE-OXALIPLATIN ,Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate),Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idelalisib, Ifex (Ifosfamide), Ifosfamide, IL-2 (Aldesleukin), Imatinib Mesylate, Imbruvica (Ibrutinib), Imiquimod, Imlygic (Talimogene Laherparepvec), Inlyta (Axitinib), Interferon Alfa-2b, Recombinant, Interleukin-2 (Aldesleukin), Intron A (Recombinant Interferon Alfa-2b), Iodine I 131 Tositumomab and Tositumomab, Ipilimumab, Tress a (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate) ,Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP,Mozobil (Plerixafor), Mustargen (Mechlorethamine Hydrochloride),,Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Netupitant and Palonosetron Hydrochloride, Neupogen (Filgrastim), Nexavar (Sorafenib Tosylate) , Nilotinib, Ninlaro (Ixazomib Citrate), Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA , Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV , Pegaspargase, Peginterferon Alfa-2b, PEG-Intron (Peginterferon Alfa-2b), Pembrolizumab, Pemetrexed Disodium Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide) , Rheumatrex (Methotrexate), Rituximab, Rolapitant Hydrochloride, Romidepsin , Romiplostim, Rubidomycin (Daunorubicin Hydrochloride), Ruxolitinib Phosphate, Sclerosol Intrapleural Aerosol (Talc),Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarcev a (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thioguanine, Thiotepa, Tolak (Fluorouracil--Topical), Toposar (Etoposide), Topotecan Hydrochloride, Toremifene, Torisel (Temsirolimus), Tositumomab and Iodine I 131, Tositumomab, Totect (Dexrazoxane Hydrochloride), TPF, Trabectedin, Trametinib, Trastuzumab, Treanda (Bendamustine Hydrochloride) , Trifluridine and Tipiracil Hydrochloride, Trisenox (Arsenic Trioxide), Tykerb (Lap atinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI,XELOX, Xgeva (Denosumab), Xofigo (Radium 223 Dichloride), Xtandi (Enzalutamide), Yervoy (Ipilimumab),Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

### Methods of Preparation

Some embodiments relate to the preparation of the compound of formula (II-A), comprising

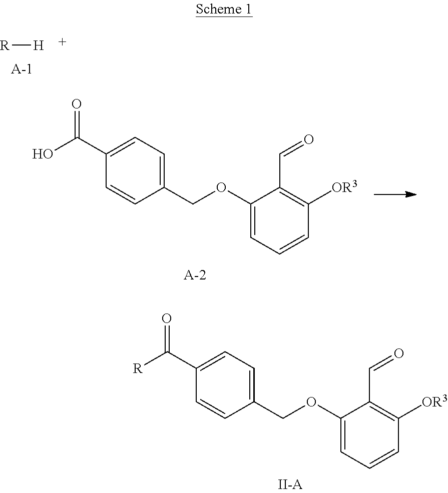

reacting a compound of formula (A-1) with a compound of formula A-2. In some embodiments, the compound of formula (A-1) is an amino acid. In some embodiments, the compound of formula (II-A) is a amino acid residue, such as an amino acid residue selected from Gly, Ala, Phe, Tyr, Glu, Leu, Ser, Arg, Gln, Val, Lys, Thr, Asn, Met, Cys, Trp, Asp, His, Pro, or Ile. In some embodiments, R³ is H. In some embodiments, the compound of formula (A-1) is selected from the group consisting of amino acids, amino monosaccharides, alcohols, or hydroxylamine. In some embodiments, the compound of formula (A-1) is D-glucosamine, alcohol (e.g., methanol), or hydroxylamine. In some embodiments, R is a radical formed upon deprotonation of R—H.

Some embodiments relate to the preparation of the compound of formula (II-B), comprising: reacting tucaresol with NH₂-OR^{a}. In some embodiments, R^{a} is H or C₁₋₆ alkyl.

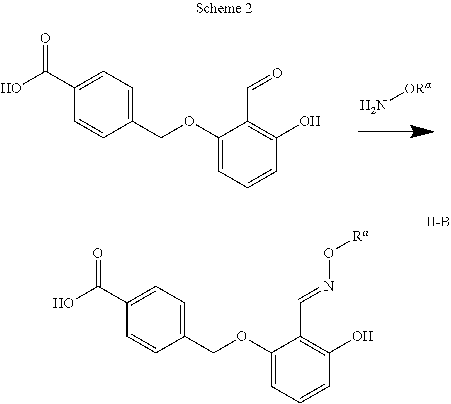

Some embodiments relate to the preparation of compound of formula (II-C), comprising protecting the carboxylic acid group on tucaresol to form a compound of formula (A-3), converting the hydroxyl group in the compound of formula (A-3) to an esater and then undergoing hydrolysis to form the compound of formula (II-C). In some embodiments, Rb is an alkyl. In some embodiments, Rb is methyl, ethyl, or propyl. In some embodiments, Rb is methyl. In some embodiments, R′ is a 5-6 membered heterocyclyl containing one oxygen heteroatom, substituted with up to four substituents each independently selected from —OH, —OC(O)CH₃, —CH₂OH, —CH₂OC(O)CH₃, —(C₂₋₃ alkylene)-OH, and —(C₂₋₃ alkylene)-OC(O)CH₃. In some embodiments, R′ is a 5-6 membered monosaccharide ring, selected from the group consisting of D-glucose, 2-deoxy-D-glucose, D-ribose, and 2-deoxy-D-ribose. In some embodiments, R′ is acyl, D-glucose, 2-deoxy-D-glucose, D-ribose, or 2-deoxy-D-ribose. In some embodiments, R′ is

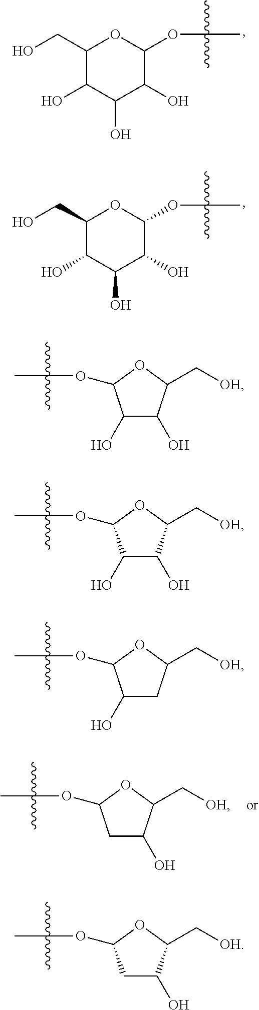

In some embodiments, R′ is

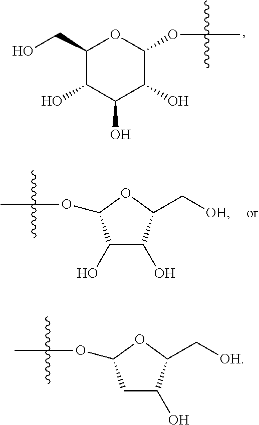

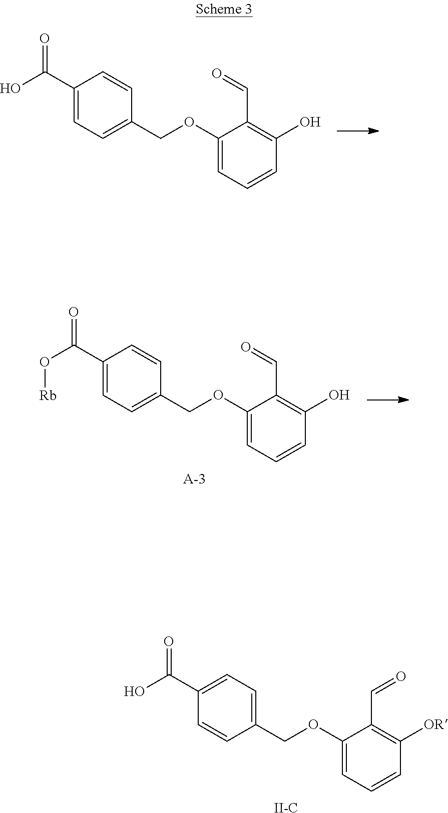

The compounds disclosed herein may be synthesized by methods described above, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry(ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples.

### EXAMPLES

### Example 1

TABLE 1
Exemplary Synthesis of Tucaresol Prodrugs

| Com- | |
|---|---|
| pound | |
| No. | Exemplary Synthetic Scheme |
| A01 | 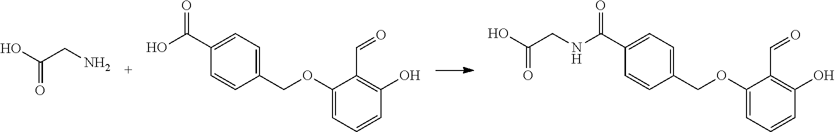 [SMILES: NCC(=O)O ; O=Cc1c(O)cccc1OCc1ccc(C(=O)O)cc1 ; O=Cc1c(O)cccc1OCc1ccc(C(=O)NCC(=O)O)cc1] |
| | A01 |
| A02 | 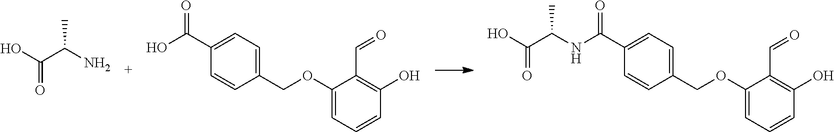 |
| | A02 |
| A03 | 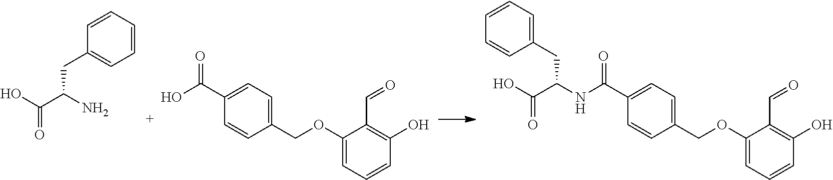 [SMILES: N[C@@H](Cc1ccccc1)C(=O)O ; O=Cc1c(O)cccc1OCc1ccc(C(=O)O)cc1 ; O=Cc1c(O)cccc1OCc1ccc(C(=O)N[C@@H](Cc2ccccc2)C(=O)O)cc1] |
| | A03 |
| A04 | 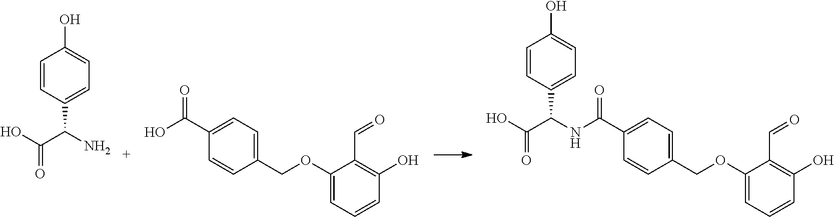 [SMILES: N[C@H](C(=O)O)c1ccc(O)cc1 ; O=Cc1c(O)cccc1OCc1ccc(C(=O)O)cc1 ; O=Cc1c(O)cccc1OCc1ccc(C(=O)N[C@H](C(=O)O)c2ccc(O)cc2)cc1] |
| | A04 |
| A05 | 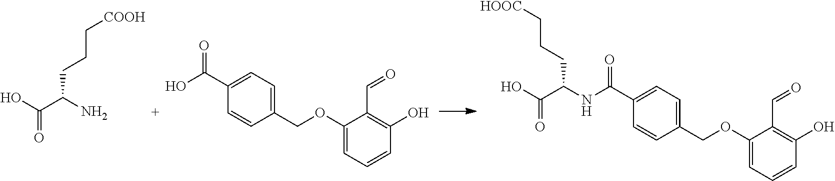 [SMILES: N[C@@H](CCCC(=O)O)C(=O)O ; O=Cc1c(O)cccc1OCc1ccc(C(=O)O)cc1 ; O=Cc1c(O)cccc1OCc1ccc(C(=O)N[C@@H](CCCC(=O)O)C(=O)O)cc1] |
| | A05 |
| A06 | 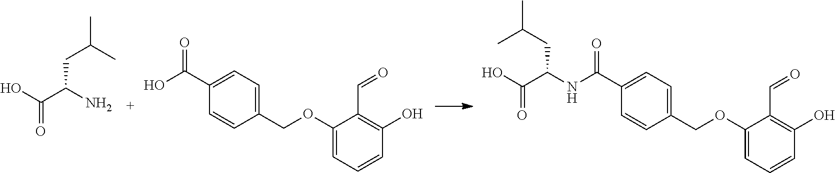 |
| | A06 |
| A07 | 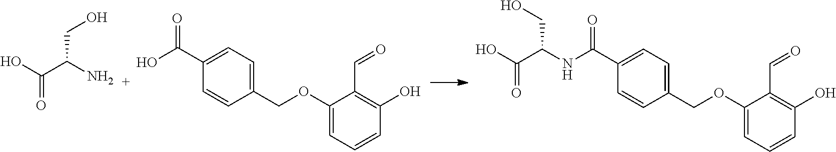 [SMILES: N[C@@H](CO)C(=O)O ; O=Cc1c(O)cccc1OCc1ccc(C(=O)O)cc1 ; O=Cc1c(O)cccc1OCc1ccc(C(=O)N[C@@H](CO)C(=O)O)cc1] |
| | A07 |
| A08 | 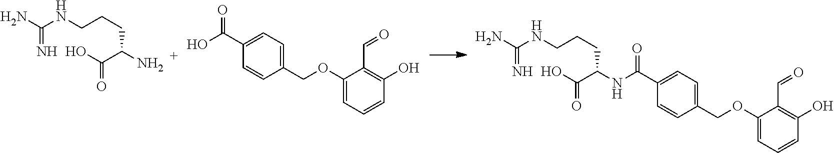 [SMILES: N=C(N)NCCC[C@H](N)C(=O)O ; O=Cc1c(O)cccc1OCc1ccc(C(=O)O)cc1 ; N=C(N)NCCC[C@H](NC(=O)c1ccc(COc2cccc(O)c2C=O)cc1)C(=O)O] |
| | A08 |
| A09 | 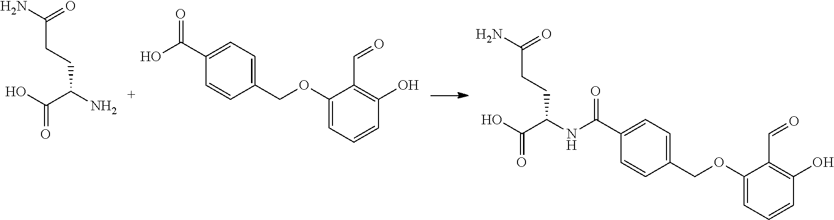 [SMILES: NC(=O)CC[C@H](N)C(=O)O ; O=Cc1c(O)cccc1OCc1ccc(C(=O)O)cc1 ; NC(=O)CC[C@H](NC(=O)c1ccc(COc2cccc(O)c2C=O)cc1)C(=O)O] |
| | A09 |
| A10 | 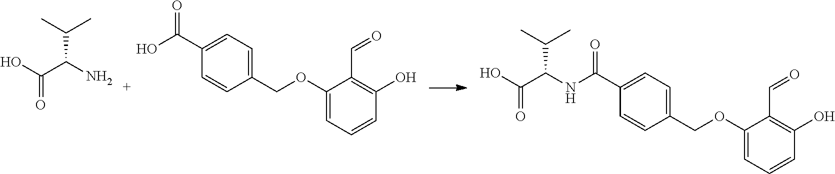 |
| | A10 |
| A11 | 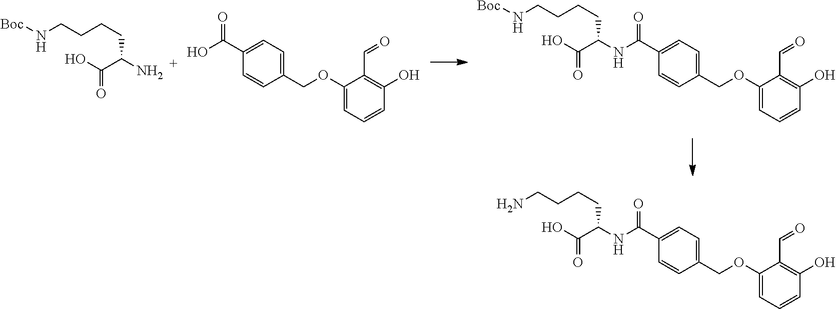 [SMILES: CC(C)(C)OC(=O)NCCCC[C@H](N)C(=O)O ; O=Cc1c(O)cccc1OCc1ccc(C(=O)O)cc1 ; CC(C)(C)OC(=O)NCCCC[C@H](NC(=O)c1ccc(COc2cccc(O)c2C=O)cc1)C(=O)O ; NCCCC[C@H](NC(=O)c1ccc(COc2cccc(O)c2C=O)cc1)C(=O)O] |
| | A11 |
| A12 | 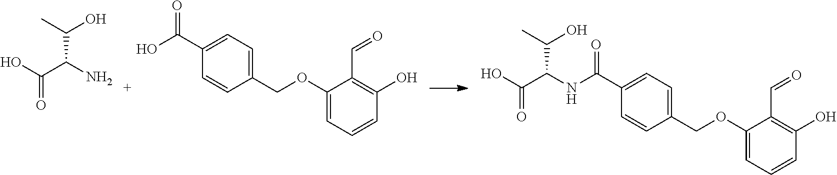 |
| | A12 |
| A13 | 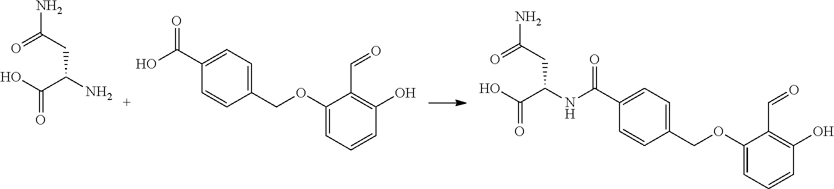 [SMILES: NC(=O)C[C@H](N)C(=O)O ; O=Cc1c(O)cccc1OCc1ccc(C(=O)O)cc1 ; NC(=O)C[C@H](NC(=O)c1ccc(COc2cccc(O)c2C=O)cc1)C(=O)O] |
| | A13 |
| A14 | 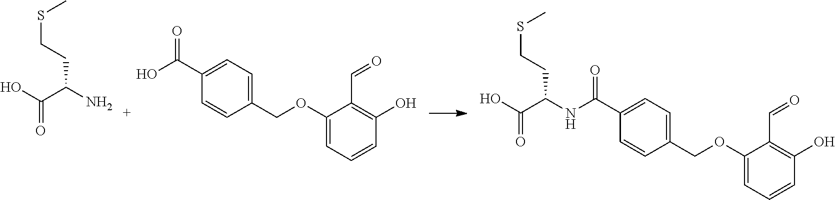 |
| | A14 |
| A15 | 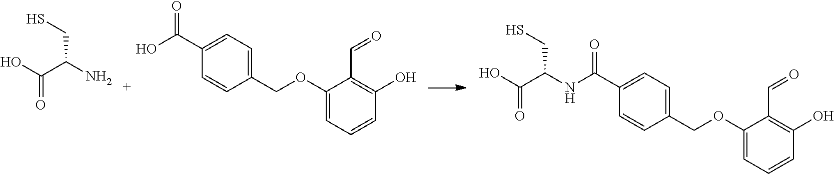 [SMILES: N[C@@H](CS)C(=O)O ; O=Cc1c(O)cccc1OCc1ccc(C(=O)O)cc1 ; O=Cc1c(O)cccc1OCc1ccc(C(=O)N[C@@H](CS)C(=O)O)cc1] |
| | A15 |
| A16 | 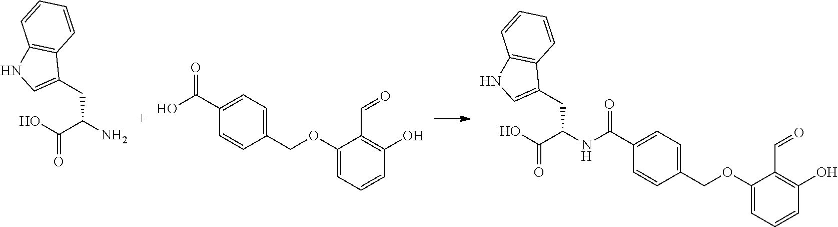 [SMILES: N[C@@H](Cc1c[nH]c2ccccc12)C(=O)O ; O=Cc1c(O)cccc1OCc1ccc(C(=O)O)cc1 ; O=Cc1c(O)cccc1OCc1ccc(C(=O)N[C@@H](Cc2c[nH]c3ccccc23)C(=O)O)cc1] |
| | A16 |
| A17 | 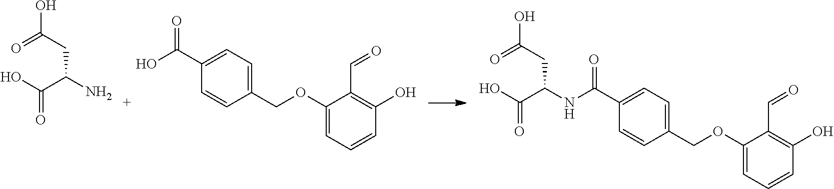 [SMILES: N[C@@H](CC(=O)O)C(=O)O ; O=Cc1c(O)cccc1OCc1ccc(C(=O)O)cc1 ; O=Cc1c(O)cccc1OCc1ccc(C(=O)N[C@@H](CC(=O)O)C(=O)O)cc1] |
| | A17 |
| A18 | 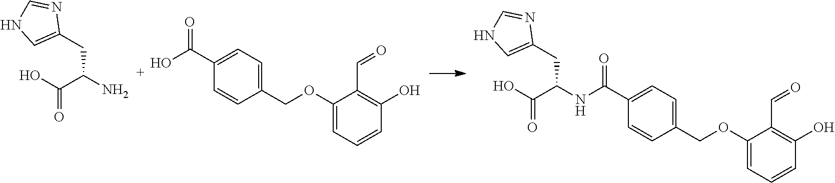 [SMILES: N[C@@H](Cc1c[nH]cn1)C(=O)O ; O=Cc1c(O)cccc1OCc1ccc(C(=O)O)cc1 ; O=Cc1c(O)cccc1OCc1ccc(C(=O)N[C@@H](Cc2c[nH]cn2)C(=O)O)cc1] |
| | A18 |
| A19 | 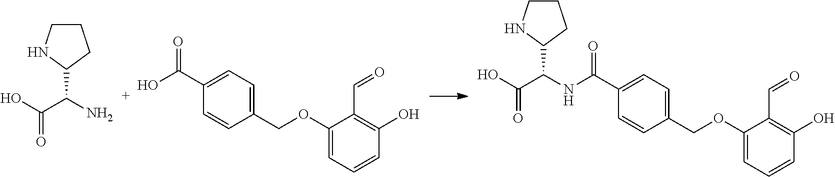 [SMILES: N[C@H](C(=O)O)C1CCCN1 ; O=Cc1c(O)cccc1OCc1ccc(C(=O)O)cc1 ; O=Cc1c(O)cccc1OCc1ccc(C(=O)N[C@H](C(=O)O)C2CCCN2)cc1] |
| | A19 |
| A20 | 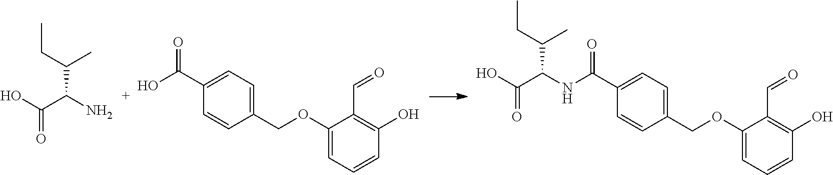 |
| | A20 |
| A21 | 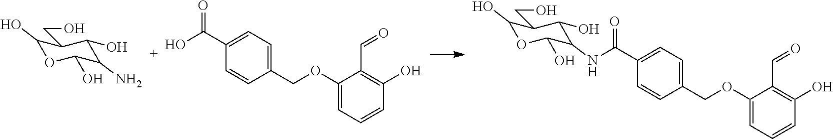 [SMILES: NC1C(O)OC(O)C(CO)C1O ; O=Cc1c(O)cccc1OCc1ccc(C(=O)O)cc1 ; O=Cc1c(O)cccc1OCc1ccc(C(=O)NC2C(O)OC(O)C(CO)C2O)cc1] |
| | A21 |
| A22 | 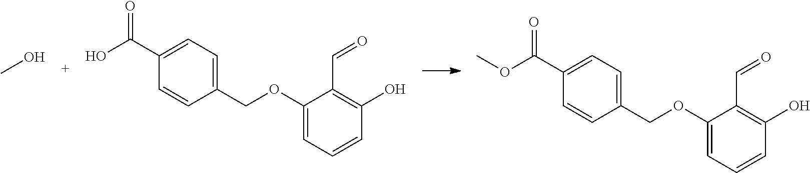 |
| | A22 |
| B01 | 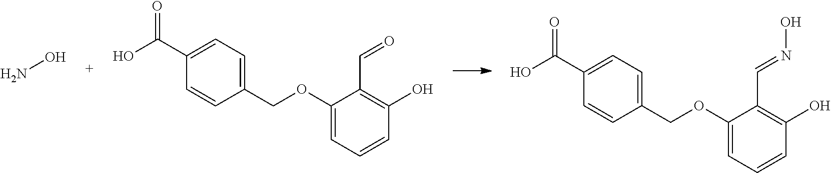 |
| | B01 |
| C03 | 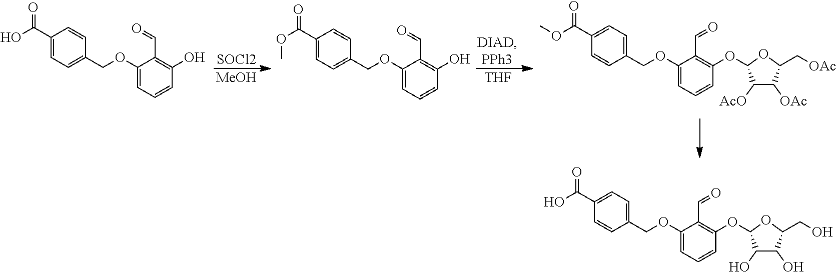 |
| | C03 |
| C04 | 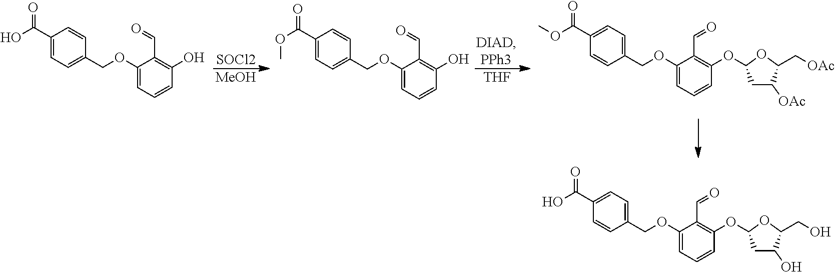 |
| | C04 |

Tucaresol reacted with each corresponding amino acid to form Compounds A01 to A20, respectively.

Tucaresol reacted with D-glucosamine, methanol and hydroxylamine, respectively, to form Compounds A21, A22 and B01.

With respect to Compounds C03 and C04, initially, methyl ester of tucaresol was obtained by adding tucaresol into methanol, cooling the solution to 0° C. and adding SOC12. The methyl ester was then added to a mixture of PPh3 and THF and reacted with DIAD to obtain Compounds C03 and C04, respectively.

### Example 2

### Compound B02:

### (E)−4+((3-hydroxy-2-((methoxyimino)methyl)phenoxy)methyl)benzoic acid

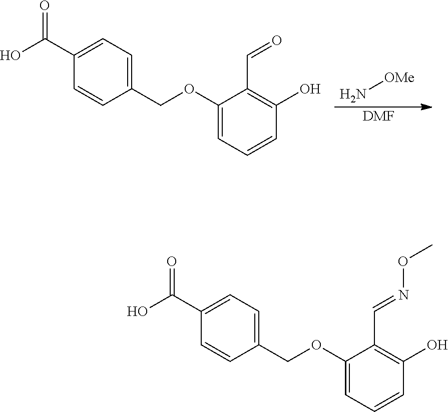

Tucaresol (8.5 g, 31 mmol, 1.0 eq), K₂CO₃ (13.8g, 100 mmol, 3 eq) and 70 ml DMF were added to a 250 ml flask. Methoxyarnine hydrochloride (4.8 g, 58 mmol. 1.7 eq) was subsequently added in portions. The mixture was then stirred for 8 h at 25° C. and poured to 200 ml water, which was acidified by HC1 to pH 1-2. The resulting solid was filtered and dried in oven at 50° C. The crude product (8.6 g) was refluxed in 60 ml ethyl acetate, cooled to room temperature and filtered to obtain 7.1 g white solid (80% yield). H NMR (in d6 DMSO): 7.863 (1H,S), 7.270-7.250 (2H, d), 6.755-6.736 (2H, d), 6.409-6.406(1H, t), 5.728-5.736 (2H,t) ,4.413 (2H,S), 3.16 (3H, s). TLC DCM:MeOH=20:1, Rf=0.6.

### Example 3

### Compound B02-1:

### ethyl(E)-(4-((3-hydroxy-2-((methoxyimino)methyl)phenoxy)methyl)benzoyl)glycinate

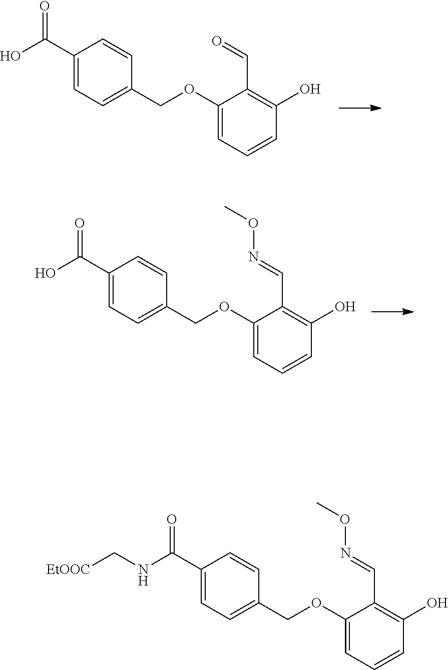 [SMILES: O=Cc1c(O)cccc1OCc1ccc(C(=O)O)cc1 ; CO/N=C/c1c(O)cccc1OCc1ccc(C(=O)O)cc1 ; CCOC(=O)CNC(=O)c1ccc(COc2cccc(O)c2/C=N/OC)cc1]

Compound B02 (1.7g, 5mmo1, 1.0eq), glycinemethylester hydrochloride (0.81g, 6 mmo1, 1.2 eq), HOBT (0.81 g, 6 mmo1, 1.2 eq) and 20 ml THF were added into a 100 ml three-necked flask. After TEA (2.5 g, 5.0 eq) was added dropwise, the solution was stirred for 2 h at 25oC. The reaction mixture was then added to 100 ml of water, which was extracted with 50 ml DCM. Subsequently, the DCM solution was dried (Na2SO4) and distilled under reduced pressure. The dry, crude mixture was purified by column separation. The fraction of Rf=0.5-0.6 (PE/EA=1/1) was collected and distilled to obtain 250 mg of title compound (13% yield). H NMR (in CDC13): 10.36 (1 H, S); 8.7 (1H, S); 7.85-7.87 (2H, d), 7.49-7.51 (2H, m); 7.1 (1H, m); 5.15 (2 H, S); 4.26-4.32 (4H, m); 4.00 (3H, S), 1.33-1.37 (3H, t).

### Example 4

### Compound C01:

### 4-((3-acetoxy-2-formylphenoxy)methyl)benzoic acid

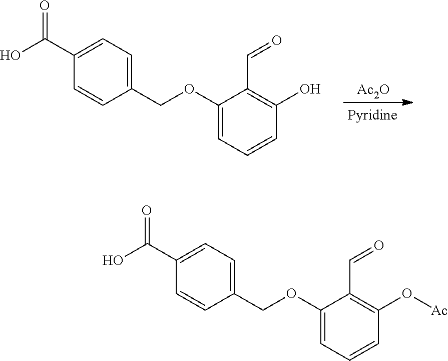 [SMILES: "Ac2O" ; O=Cc1c(O)cccc1OCc1ccc(C(=O)O)cc1 ; CC(=O)Oc1cccc(OCc2ccc(C(=O)O)cc2)c1C=O]

Tucaresol (3.0 g, 11 mmol, 1.0 eq) and 30 ml pyridine was added to a 100 ml flask, which was pre-cooled to 0° C. After acetate anhydride was added slowly, the solution was warmed to 25° C. and stirred for 72 h. Solvent was then removed and purified by column purification. The fraction of Rf=0.4 (PE/EA/Ac=1/2/drops) was collected to obtain 0.64 g title compound. H NMR (in d6 DMSO): 12.93 (1H,S), 10.395 (1H, S); 8.00 (1H, d); 7.629-7.696 (3H, m); 7.254 (1H, d); 6.831 (1H, d); 5.763 (2H, s), 2.282 (3H, s).

### Example 5

### Compound C02-1:

### methyl 4-((2-formyl-3-hydroxyphenoxy)methyl)benzoate

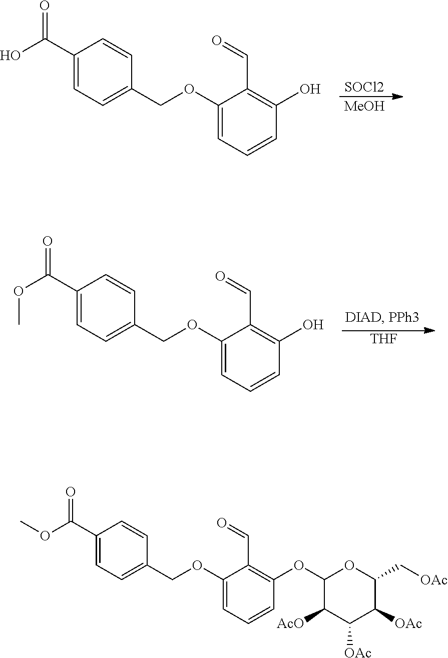

Tucaresol (10.0 g, 36 mmol, 1.0 eq) and 100 ml methanol were added to a 250 ml flask. After the solution was cooled to 0° C., 4 ml of SOC12 was added carefully. The reaction solution was refluxed for 5 h. Subsequently, the solution was distilled to dry, slurried in PE/EA (10/1) and filtered to obtain 9.4 g of title compound (Rf=0.55, EA/PE=2/1), which was used directly without further purification.

The obtained methyl ester of Tucaresol (9.4 g, 32 mmol, 1.0 eq) was added to a 250 ml flask together with (2R,3R,4S,5R)-2-(acetoxymethyl)-6-hydroxytetrahydro-2H-pyran-3,4,5-triyl triacetate (12 g, 36 mmol, 1.1 eq), PPh3 (10 g, 40 mmol, 1.2 eq) and 150 ml THF. After the reaction solution was cooled to 0° C., 20 ml of DIAD was added carefully to keep the temperature below 5° C. Once reaction completed, 300 ml of water was added. 200 ml of EA was added to extract the resulting compound from the aqueous solution. The solvent was then removed. And the resulting compound was purified by column. The fraction of Rf=0.7 (EA:PE=2:1) was collected and distilled to remove solvent. 3.0 g of title compound was obtained. H NMR (CDC13) 10.473 (1H, s), 8.090 (2H, d), 7.577 (2H, d), 7.404 (1H, t), 6.750 (2H, m), 5.144-5.373 (5H, m), 4.297 (1H, m), 4.230 (1H,m), 3.941 (3H, s), 3.932 (1H, m), 2.045-2.180 (12 H, m).

### Example 6

### Compound C02

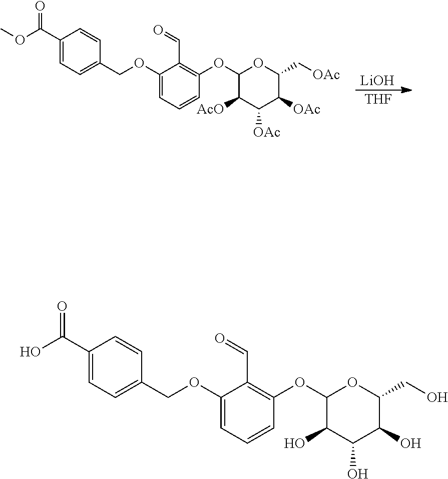

Compound C02-1 (2.0 g, 33 mmol, 1.0 eq), 20 ml THF and 20 mol H₂O were added to a 100 ml flask. After the solution was cooled to 0° C., LiOH.H₂O (8 g, 200 mmol) was added. The reaction solution was stirred at 25° C. for 16 h and, subsequently, cooled to 0° C. and acidified by 1 M HC1 to pH=2. The resulting solid was filtered and purified by column. The fraction of Rf=0.1 (DCM/Methanol/Ac=10/1/drops) was collected and solvent was removed to obtain 0.7 g title compound. H NMR: (DMSO) 12.928 (1H s), 10.516 (1H s), 7.985 (2H d), 7.650 (2H d), 7.558 (1H t), 4.943-5.421 (6H m), 4.608 (1H s), 3.179-3.717 (6H m).

### Example 7

### Intravenous and Oral Dosing Pharmacokinetic Screen for Tucaresol, and for Compounds B02, C01, C02-1 and C02

Tucaresol, Compound B02, Compound C01, Compound C02-1, and Compound C02 were dosed once, by IV bolus (3 mg/kg in 20% hydroxyprophyl-13-cyclodextrin with 1 eq NaOH) or PO (10 mg/kg in 1% carboxymethylcellulose/0.1% tween-80 in water), to non-fasted male C57BL/6 mice. Animals were bled from the dorsal metatarsal vein (from the heart for 24 hour time point) for K2-EDTA plasma 0.083 (for IV only), 0.25, 0.5, 1, 2, 4, 8, and 24 hours after dosing. The plasma concentration of the dosed tucaresol or compound were measured by LC/MS/MS (I-CMS-8060 instrument for MS). PK parameters were then calculated with WinNonlin 6.1. For Compounds C01, CO2 and CO2-1, stability in C57BL/6 K2EDTA plasma was also evaluated at 22° C. over 1 hour by utilizing LC.

TABLE 2
Plasma PK parameters of Tucaresol or Compounds following IV (3 mg/kg) or PO (10 mg/kg)
dosing of Tucaresol, Compounds B02, C01, C02-1 and C02

| | | | | Comp. | | Comp. | | Comp. | | Comp. | |
|---|---|---|---|---|---|---|---|---|---|---|---|
| PK parameter | Units | Tucaresol | SD | B02 | SD | C01 | SD | C02 | SD | C02-1 | SD |
| T_{1/2} | h | 4.67 | 0.09 | 5.49 | 1.14 | 4.24 | 0.36 | 3.94 | 0.39 | 13.8 | 13.1 |
| Tₘₐₓ | h | 0.250 | 0.000 | 5.42 | 4.47 | 0.333 | 0.14 | 0.500 | NA | 3.33 | 1.15 |
| Cₘₐₓ | ng/Ml | 3038 | 169 | 32.3 | 4.7 | 645 | 80 | 1398 | 201 | 121 | 24 |
| AUCₗₐₛₜ | h*ng/Ml | 8252 | 432 | 219 | 127 | 2802 | 157 | 5933 | 439 | 709 | 149 |
| AUC_{lnf} | h*ng/Ml | 8427 | 453 | 356 | 36 | 2848 | 155 | 6004 | 448 | 2323 | 1321 |
| AUC__{%Extrap}_obs | % | 2.08 | 0.14 | 26.7 | 30.8 | 1.64 | 0.56 | 1.17 | 0.40 | 61.4 | 22.1 |
| MRT_{Inf}_obs | h | 4.45 | 0.19 | 10.0 | 0.6 | 5.29 | 0.31 | 5.17 | 0.38 | 21.5 | 18.8 |
| AUCₗₐₛₜ/D | h*mg/mL | 825 | 43 | 24.3 | 14.0 | 4.24 | 0.36 | 947 | 70 | 161 | 34 |
| F | % | 75.0 | 4.0 | NA | NA | 0.333 | NA | NA | NA | NA | NA |

Mean (n = 3)

TABLE 3
Plasma PK measurements of Tucaresol following
IV or PO dosing of Tucaresol

| | Tucaresol concentration (ng/mL) | | | Mean | SD | CV |
|---|---|---|---|---|---|---|
| Time (h) | Mouse 1 | Mouse 2 | Mouse 3 | (ng/mL) | (ng/mL) | (%) |

IV Dose: 3 mg/kg

| | | | | | | |
|---|---|---|---|---|---|---|
| 0.083 | 1355 | 2053 | 2275 | 1894 | 480 | 25.4 |
| 0.25 | 1239 | 1267 | 1475 | 1327 | 129 | 9.74 |
| 0.5 | 972 | 1007 | 976 | 985 | 19 | 1.95 |
| 1 | 623 | 704 | 794 | 707 | 86 | 12.1 |
| 2 | 473 | 363 | 428 | 421 | 56 | 13.2 |
| 4 | 122* | 113* | 110* | 115 | 7 | 5.76 |
| 8 | 73.6* | 81.1* | 65.6* | 73.4 | 7.8 | 10.6 |
| 24 | 12.0* | 9.48* | 6.96* | 9.5 | 2.5 | 26.8 |

PO Dose: 10 mg/kg

| | | | | | | |
|---|---|---|---|---|---|---|
| 0.25 | 2930 | 3233 | 2951 | 3038 | 169 | 5.57 |
| 0.5 | 2770 | 2392 | 2076 | 2413 | 347 | 14.4 |
| 1 | 1867 | 1680 | 1696 | 1748 | 103 | 5.91 |
| 2 | 822 | 921 | 902 | 881 | 53 | 5.96 |
| 4 | 603* | 626* | 614* | 614 | 12 | 1.88 |
| 8 | 185* | 222* | 164* | 190 | 29 | 153 |
| 24 | 26.3* | 28.5* | 23.2* | 26.0 | 2.7 | 10.2 |

*Data included in terminal elimination T½

TABLE 4
Plasma PK parameters of Compound B02 and Tucaresol
following IV or PO dosing of Compound B02 and tucaresol

| | B02 concentration (ng/mL) | | | Mean | SD | |
|---|---|---|---|---|---|---|
| Time (h) | Mouse 1 | Mouse 2 | Mouse 3 | (ng/mL) | (ng/mL) | CV (%) |

IV Dose: 3 mg/kg Compound B02

| | | | | | | |
|---|---|---|---|---|---|---|
| 0.083 | 3215 | 3036 | 2891 | 3047 | 162 | 5.32 |
| 0.25 | 1745 | 1157 | 1108 | 1337 | 355 | 26.5 |
| 0.5 | 335 | 271 | 242 | 283 | 47 | 16.7 |
| 1 | 87.7 | 34.9 | 22.3 | 48.3 | 34.7 | 71.8 |
| 2 | 179* | 194* | 189* | 187 | 8 | 4.08 |
| 4 | 43.4* | 66.8* | 51.7* | 54.0 | 11.8 | 21.9 |
| 8 | 11.2* | 15.8* | 27.1* | 18.0 | 8.2 | 45.2 |
| 24 | BLOQ | BLOQ | BLOQ | NA | NA | NA |

PO dose: 10 mg/kg Compound B02

| 0.25 | 5784 | 3293 | 3697 | 4258 | 1337 | 31.4 |
|---|---|---|---|---|---|---|
| 0.5 | 3368 | 1901 | 2669 | 2646 | 734 | 27.7 |
| 1 | 1064 | 395 | 564 | 674 | 348 | 51.6 |
| 2 | 876 | 257 | 301 | 478 | 345 | 72.2 |
| 4 | 468* | 432* | 336* | 412 | 68 | 16.5 |
| 8 | 114* | 146* | 196* | 152 | 41 | 27.3 |
| 24 | 6.93* | 11.6* | 12.9* | 10.5 | 3.1 | 29.9 |
| | Tucaresol plasma | | | | | |
| | concentration (ng/mL) | | | Mean | SD | |
| Time (h) | Mouse 1 | Mouse 2 | Mouse 3 | (ng/mL) | (ng/mL) | CV (%) |

IV dose: Tucaresol plasma concentration data

| | | | | | | |
|---|---|---|---|---|---|---|
| 0.083 | 12.9 | 12.8 | 11.7 | 12.5 | 0.7 | 5.24 |
| 0.25 | 7.47 | 4.22 | 6.34 | 6.01 | 1.65 | 27.5 |
| 0.5 | BLOQ | BLOQ | BLOQ | NA | NA | NA |
| 1 | BLOQ | 4.02 | BLOQ | NA | NA | NA |
| 2 | BLOQ | 4.47 | 4.12 | 4.30 | NA | NA |
| 4 | 8.31 | 6.13 | 6.70 | 7.04 | 1.13 | 16.1 |
| 8 | 7.15 | 8.78 | 9.64 | 8.52 | 1.27 | 14.9 |
| 24 | 2.65 | BLOQ | BLOQ | NA | NA | NA |

PO dose: Tucaresol plasma concentration data

| | | | | | | |
|---|---|---|---|---|---|---|
| 0.25 | 29.3 | 10.9 | 5.47 | 16.2 | 12.5 | 82.1 |
| 0.5 | 15.7 | 5.73 | 6.62 | 9.3 | 5.5 | 58.8 |
| 1 | 8.85 | 3.01 | 4.75 | 5.54 | 3.00 | 54.1 |
| 2 | 21.7 | 5.04 | 6.37 | 11.0 | 9.2 | 83.8 |
| 4 | 27.5 | 8.98 | 10.5 | 15.7 | 10.3 | 65.6 |
| 8 | 17.7 | 29.9 | 37.7 | 28.4 | 10.0 | 35.4 |
| 24 | BLOQ | 2.80 | BLOQ | NA | NA | NA |

*Data included in terminal elimination T½
BLOQ = Below the Limit of Quantification of 2 ng/ml, NA = Not Available

TABLE 5
Plasma PK parameters of Compound C01 and Compound B02 following IV
or PO dosing of Compound C01 and Compound B02

| | Concentration (ng/mL) | | | Mean | SD | |
|---|---|---|---|---|---|---|
| Time (h) | Mouse 1 | Mouse 2 | Mouse 3 | (ng/mL) | (ng/mL) | CV (%) |

Compound C01 IV Dose: 3 mg/kg

| | | | | | | |
|---|---|---|---|---|---|---|
| 0.083 | NA | NA | NA | NA | NA | NA |
| 0.25 | NA | NA | NA | NA | NA | NA |
| 0.5 | NA | NA | NA | NA | NA | NA |
| 1 | NA | NA | NA | NA | NA | NA |
| 2 | NA | NA | NA | NA | NA | NA |
| 4 | NA | NA | NA | NA | NA | NA |
| 8 | NA | NA | NA | NA | NA | NA |
| 24 | NA | NA | NA | NA | NA | NA |

Compound C01 PO Dose: 10 mg/kg

| | | | | | | |
|---|---|---|---|---|---|---|
| 0.25 | NA | NA | NA | NA | NA | NA |
| 0.5 | NA | NA | NA | NA | NA | NA |
| 1 | NA | NA | NA | NA | NA | NA |
| 2 | NA | NA | NA | NA | NA | NA |
| 4 | NA | NA | NA | NA | NA | NA |
| 8 | NA | NA | NA | NA | NA | NA |
| 24 | NA | NA | NA | NA | NA | NA |

Compound B02 IV Plasma concentration-time data

| | | | | | | |
|---|---|---|---|---|---|---|
| 0.083 | 1057 | 1023 | 982 | 1021 | 38 | 3.68 |
| 0.25 | 529 | 759 | 784 | 691 | 141 | 20.36 |
| 0.5 | 428 | 496 | 471 | 465 | 35 | 7.45 |
| 1 | 289 | 327 | 333 | 316 | 24 | 7.52 |
| 2 | 187 | 214* | 204* | 201 | 14 | 6.85 |
| 4 | 118* | 115* | 113* | 116 | 3 | 2.21 |
| 8 | 68.9* | 61.7* | 52.0* | 60.9 | 8.5 | 14.0 |
| 24 | 5.13* | BLOQ | BLOQ | NA | NA | NA |

Compound B02 PO Plasma concentration-time data

| | | | | | | |
|---|---|---|---|---|---|---|
| 0.25 | 555 | 718 | 657 | 643 | 82 | 12.8 |
| 0.5 | 560 | 517 | 458 | 512 | 51 | 9.95 |
| 1 | 352 | 455 | 350 | 386 | 60 | 15.6 |
| 2 | 299* | 352* | 254* | 302 | 49 | 16.2 |
| 4 | 188* | 199* | 187* | 191 | 7 | 3.43 |
| 8 | 110* | 107* | 105* | 107 | 2 | 2.06 |
| 24 | 5.48* | 7.92* | 9.11* | 7.50 | 1.85 | 24.7 |

BLOQ = Below the Liimit of Quantification of 2 ng/ml, NA = Not Available
*Data included in terminal elimination T1/2

TABLE 6
Plasma PK parameters of Compound C02 and Tucaresol
following IV or PO dosing of Compound C02 and Tucaresol
Compound C02-IV Dose: 3 mg/kg

| | concentration (ng/mL) | | | Mean | SD | |
|---|---|---|---|---|---|---|
| Time (h) | Mouse 1 | Mouse 2 | Mouse 3 | (ng/mL) | (ng/mL) | CV (%) |
| 0.083 | 1826* | 2979* | 2357* | 2387 | 577 | 24.2 |
| 0.25 | 537* | 602* | 594* | 578 | 35 | 6.08 |
| 0.5 | 130* | 241* | 289* | 220 | 82 | 37.1 |
| 1 | BLOQ | BLOQ | BLOQ | NA | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |
| 24 | BLOQ | BLOQ | BLOQ | NA | NA | NA |

Compound C02-PO Dose: 10 mg/kg

| | C02 concentration (ng/mL) | | | | | |
|---|---|---|---|---|---|---|
| Time (h) | Mouse 1 | Mouse 2 | Mouse 3 | CV (%) | | |
| 0.25 | BLOQ | BLOQ | BLOQ | NA | NA | NA |
| 0.5 | 59.1 | BLOQ | BLOQ | NA | NA | NA |
| 1 | BLOQ | BLOQ | BLOQ | NA | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |
| 24 | BLOQ | BLOQ | BLOQ | NA | NA | NA |

Tucaresol IV Dose: 3 mg/kg

| | Tucaresol concentration (ng/mL) | | | Mean | SD | |
|---|---|---|---|---|---|---|
| Time (h) | Mouse 1 | Mouse 2 | Mouse 3 | (ng/mL) | (ng/mL) | CV (%) |
| 0.083 | 24.7 | 42.3 | 17.9 | 28.3 | 12.6 | 44.4 |
| 0.25 | 119 | 145 | 121 | 128 | 14 | 11.0 |
| 0.5 | 179 | 161 | 156 | 165 | 12 | 7.40 |
| 1 | 132 | 113 | 125 | 123 | 9 | 7.68 |
| 2 | 121* | 99.4 | 121* | 114 | 12 | 10.9 |
| 4 | 66.6* | 83.3* | 78.5* | 76.1 | 8.6 | 11.3 |
| 8 | 35.4* | 37.6* | 38.2* | 37.1 | 1.5 | 4.04 |
| 24 | BLOQ | 3.39* | BLOQ | NA | NA | NA |

Tucaresol-PO Dose: 10 mg/kg

| | Tucaresol concentration (ng/mL) | | | Mean | SD | |
|---|---|---|---|---|---|---|
| Time (h) | Mouse 1 | Mouse 2 | Mouse 3 | (ng/mL) | (ng/mL) | CV (%) |
| 0.25 | 896 | 958 | 1062 | 972 | 84 | 8.66 |
| 0.5 | 1338 | 1622 | 1234 | 1398 | 201 | 14.4 |
| 1 | 942 | 949 | 854 | 915 | 53 | 5.79 |
| 2 | 610 | 402 | 586 | 532 | 114 | 21.4 |
| 4 | 332 | 305 | 532 | 390 | 124 | 31.8 |
| 8 | 211 | 268 | 245 | 241 | 29 | 12.0 |
| 24 | 8.72 | 15.3 | 12.8 | 12.3 | 3.3 | 27.1 |

*Data included in terminal elimination T½

TABLE 7
Plasma PK parameters of Compound C02-1 and Tucaresol
following IV or PO dosing of Compound C02-1 and Tucaresol

| | Concentration (ng/mL) | | | Mean | SD | CV |
|---|---|---|---|---|---|---|
| Time (h) | Mouse 1 | Mouse 2 | Mouse 3 | (ng/mL) | (ng/mL) | (%) |

Compound C02-1 IV Dose: 3 mg/kg

| | | | | | | |
|---|---|---|---|---|---|---|
| 0.083 | BLOQ | BLOQ | BLOQ | NA | NA | NA |
| 0.25 | BLOQ | BLOQ | BLOQ | NA | NA | NA |
| 0.5 | BLOQ | BLOQ | BLOQ | NA | NA | NA |
| 1 | BLOQ | BLOQ | BLOQ | NA | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |
| 24 | BLOQ | BLOQ | BLOQ | NA | NA | NA |

C02-1 PO Dose: 10 mg/kg

| 0.083 | BLOQ | BLOQ | BLOQ | NA | NA | NA |
|---|---|---|---|---|---|---|
| 0.5 | BLOQ | BLOQ | BLOQ | NA | NA | NA |
| 1 | BLOQ | BLOQ | BLOQ | NA | NA | NA |
| 2 | BLOQ | BLOQ | BLOQ | NA | NA | NA |
| 4 | BLOQ | BLOQ | BLOQ | NA | NA | NA |
| 8 | BLOQ | BLOQ | BLOQ | NA | NA | NA |
| 24 | BLOQ | BLOQ | BLOQ | NA | NA | NA |
| | Tucaresol concentration (ng/mL) | | | Mean | SD | |
| Time (h) | Mouse 1 | Mouse 2 | Mouse 3 | (ng/mL) | (ng/mL) | CV (%) |

Tucaresol IV Dose: 3 mg/kg

| | | | | | | |
|---|---|---|---|---|---|---|
| 0.083 | 17.8 | 12.8 | 21.2 | 17.3 | 4.2 | 24.5 |
| 0.25 | 60.8 | 52.7 | 45.0 | 52.9 | 7.9 | 14.9 |
| 0.5 | 82.3 | 66.7 | 90.8 | 79.9 | 12.2 | 15.3 |
| 1 | 64.0 | 49.8 | 67.8 | 60.5 | 9.5 | 15.7 |
| 2 | 61.1 | 36.2 | 50.5 | 49.3 | 12.5 | 25.3 |
| 4 | 30.0 | 30.0 | 48.0 | 36.0 | 10.4 | 29.0 |
| 8 | 15.7 | 17.6 | 21.4 | 18.2 | 2.9 | 15.9 |
| 24 | BLOQ | BLOQ | BLOQ | NA | NA | NA |

Tucaresol PO Dose: 10 mg/kg

| | | | | | | |
|---|---|---|---|---|---|---|
| 0.25 | BLOQ | BLOQ | BLOQ | NA | NA | NA |
| 0.5 | 13.2 | 14.0 | 20.5 | 15.9 | 4.0 | 25.2 |
| 1 | 37.4 | 41.3 | 71.1 | 49.9 | 18.4 | 36.9 |
| 2 | 83.9 | 94.9* | 131 | 103 | 25 | 24.0 |
| 4 | 126* | 73.3* | 14* | 114 | 36 | 31.7 |
| 8 | 86.9* | 78.8* | 83.4* | 83.0 | 4.0 | 4.85 |
| 24 | BLOQ | BLOQ | BLOQ | NA | NA | NA |

*Data included in terminal elimination T½

TABLE 8
Plasma stability of Compounds C01, C02-1 and C02
Compound C01 2,000 ng/mL 22°C

| Time | Analyte | | | Mean | Remaining |
|---|---|---|---|---|---|
| (min) | area | IS area | Ratio | Ratio | % |
| 60 | NA | 7096652 | NA | NA | NA |
| | NA | 7401709 | NA | | |
| 30 | NA | 8075920 | NA | NA | NA |
| | NA | 7405529 | NA | | |
| 15 | 9483 | 7320755 | 0.00130 | 0.00125 | 1.07 |
| | 9775 | 8105838 | 0.00121 | | |
| 5 | 143854 | 8332524 | 0.0173 | 0.0175 | 15.0 |
| | 144589 | 8159794 | 0.0177 | | |
| 0 | 907735 | 8458491 | 0.107 | 0.117 | 100 |
| | 1057985 | 8402930 | 0.126 | | |

Compound C02 2,000 ng/mL 22°C

| Time | Analyte | | | Mean | Remaining |
|---|---|---|---|---|---|
| (min) | area | IS area | Ratio | Ratio | % |
| 60 | 30213 | 10056147 | 0.00300 | 0.00379 | 61.7 |
| | 41381 | 9036381 | 0.00458 | | |
| 30 | 35859 | 8978298 | 0.00399 | 0.00422 | 68.7 |
| | 45304 | 10194598 | 0.00444 | | |
| 15 | 39814 | 9170048 | 0.00434 | 0.00489 | 79.6 |
| | 48146 | 8840924 | 0.00545 | | |
| 5 | 35209 | 8516732 | 0.00413 | 0.00383 | 62.3 |
| | 31534 | 8958831 | 0.00352 | | |
| 0 | 56731 | 8714371 | 0.00651 | 0.00615 | 100 |

Compound C02-1 2,000 ng/mL 22°C

| Time | Analyte | | | Mean | Remaining |
|---|---|---|---|---|---|
| (min) | area | IS area | Ratio | Ratio | % |
| 60 | 1540 | 6815869 | 0.000226 | 0.000296 | 2.37 |
| | 2045 | 5588136 | 0.000366 | | |
| 30 | 3056 | 5823331 | 0.000525 | 0.000818 | 6.56 |
| | 5223 | 4701517 | 0.00111 | | |
| 15 | 11036 | 4732052 | 0.00233 | 0.00208 | 16.7 |
| | 9068 | 4979468 | 0.00182 | | |
| 5 | 10842 | 4986700 | 0.00217 | 0.00235 | 18.9 |
| | 12564 | 4976042 | 0.00252 | | |
| 0 | 70806 | 5733541 | 0.0123 | 0.0125 | 100 |

Table 2 provides the PK parameters for tucaresol in blood after dosing tucaresol, Compound B02, Compound C01, Compound C02-1 and Compound C02, after IV (3 mg/kg) or PO (10 mg/kg). Specifically, Table 2 demonstrated that Compound C02, when dosed orally, resulted in plasma tucaresol levels similar to that when tucaresol was dosed orally. Compound C01, when dosed orally, resulted in significant but lower tucaresol exposure than Compound C02 dosing.

Tables 3 through 7 report individual measurements made on PK samples in this study.

Table 8 reports the mouse plasma stability of Compounds C01, C02, and C02-1. Compounds C01 and C02-1 are unstable at 22° C. Compound C02 showed initial instability but leveled off at approximately 60-70% of starting through 1 hour.

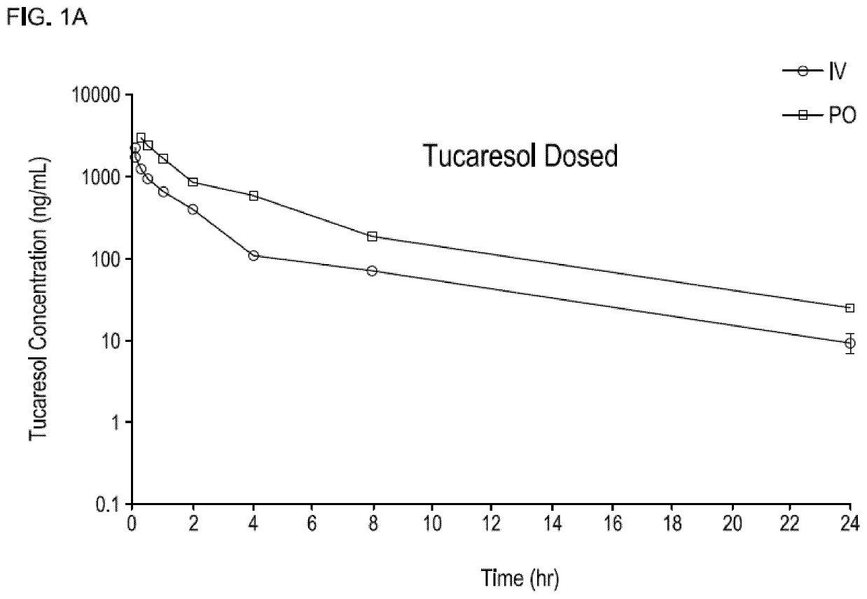

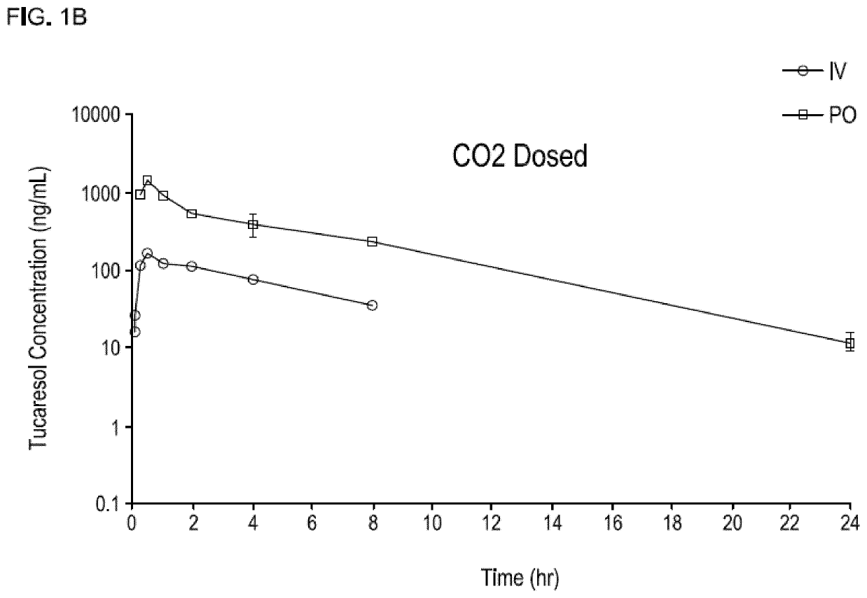

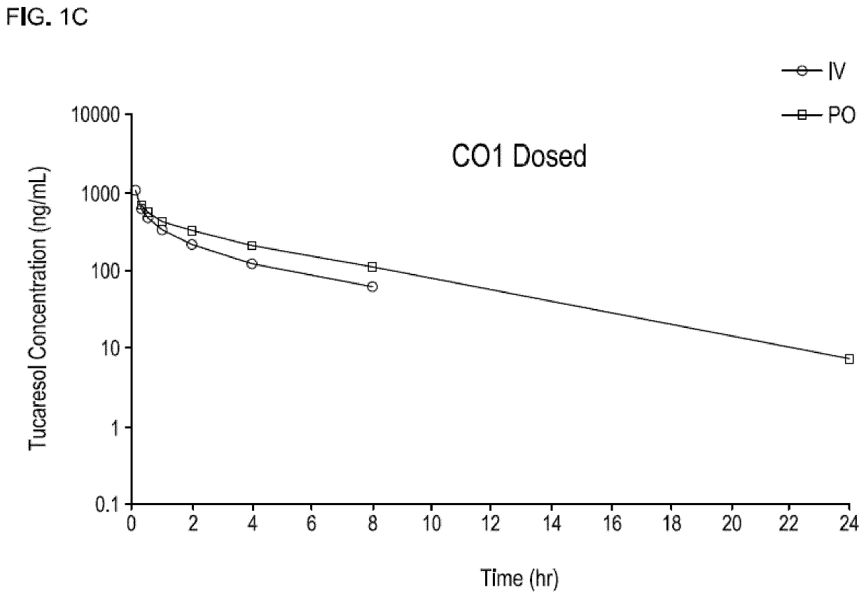

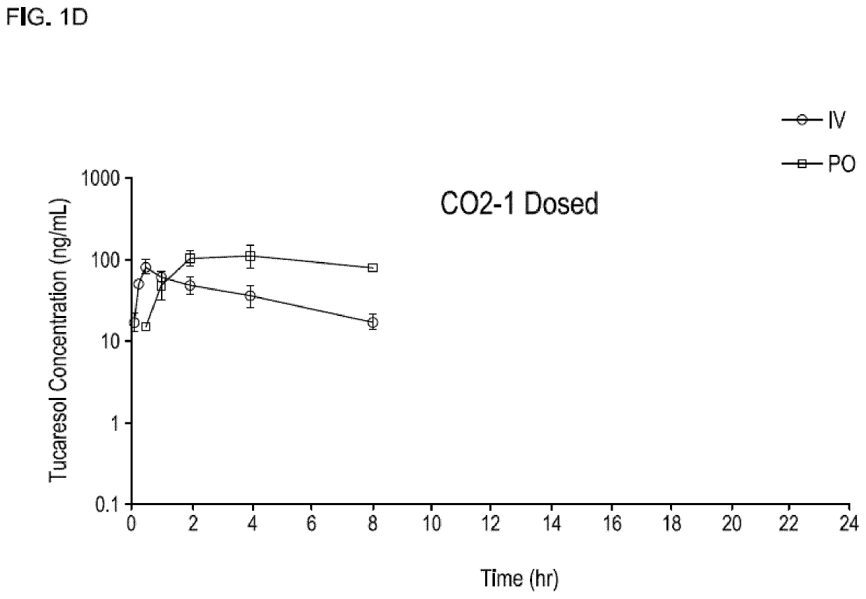

Oral dosing of Compounds C02 and C01 in C57BL/6 mice resulted in significant exposure to tucaresol in plasma, and Compound CO2 achieved similar levels to that achieved when tucaresol was dose orally. The plasma concentrations of tucaresol in the PO and IV dosing of tucaresol, compound C01, compound C02, and compound C02-01 are respectively shown in FIGS. 1A, 1B, 1C, and 1D. Table 9 shows the pharmacokinetics of tucaresol after a single dose of tucaresol in healthy subjects.

TABLE 9

| | | | Plasma | | | | |
|---|---|---|---|---|---|---|---|
| | Dose | Weight | t_{1/2,z} | CL₀ | Cₘₐₓ | tₘₐₓ | AUC |
| Subject | (mg) | (kg) | (h) | (ml min⁻¹) | (μg ml⁻¹) | (h) | (μg ml⁻¹ h) |
| 1 | 200 | 82.5 | | | 9.9 | 5.00 | |
| 2 | 200 | 77.8 | | | 11.1 | 5.00 | |
| 3 | 400 | 65.0 | 133 | 2.35 | 16.6 | 4.03 | 2829 |
| 4 | 400 | 69.0 | 190 | 1.86 | 19.6 | 3.00 | 3562 |
| 7 | 800 | 84.7 | 134 | 2.72 | 28.3 | 4.00 | 4889 |
| 8 | 800 | 68.0 | 126 | 2.31 | 36.5 | 4.08 | 5759 |
| 5 | 1200 | 80.5 | 252 | 1.65 | 40.4 | 8.00 | 12103 |
| 6 | 1200 | 74.0 | 192 | 2.17 | 40.1 | 6.00 | 9180 |
| 1 | 1200 | | 123 | 3.33 | 39.3 | 4.00 | 5987 |
| 3 | 1200 | | 215 | 2.44 | 33.7 | 8.00 | 8161 |
| Mean | | | 195 | 2.40 | 38.4 | 6.50 | 8858 |
| 2 | 2000 | | 278 | 2.04 | 60.8 | 6.00 | 16273 |
| 4 | 2000 | | 222 | 2.01 | 59.3 | 6.00 | 16487 |
| 6 | 2000 | | 207 | 2.37 | 57.1 | 6.00 | 14010 |
| 9 | 2000 | 82.0 | 209 | 2.67 | 57.1 | 4.00 | 12455 |
| Mean | | | 229 | 2.27 | 58.6 | 5.50 | 14806 |
| 7 | 2800 | | 232 | 3.61 | 56.6 | 6.00 | 12881 |
| 8 | 2800 | | 240 | 2.23 | 87.7 | 6.00 | 20871 |
| 1 | 2800 | | 178 | 2.97 | 91.4 | 6.00 | 15635 |
| 3 | 2800 | | 241 | 2.58 | 59.3 | 12.00 | 18018 |
| Mean | | | 223 | 2.85 | 73.7 | 7.50 | 16851 |
| 6 | 3600 | | 291 | 2.36 | 81.7 | 6.08 | 25353 |
| 2 | 3600 | | 329 | 2.15 | 86.3 | 8.00 | 27818 |
| 7 | 3600* | | 265 | 2.48 | 80.3 | 8.00 | 24131 |
| 8 | 3600* | | 268 | 2.05 | 134.7 | 4.00 | 29116 |
| Mean | | | 289 | 2.26 | 95.8 | 10.02 | 26604 |

### Example 8

### In Vivo Efficacy Study of Tucaresol and Compound C02 as a Single Agent in the Treatment of Subcutaneous Hepa 1-6 Mouse Liver Cancer Model

The study evaluated preclinically in vivo therapeutic efficacy of tucaresol and Compound C02 as a single agent in the treatment of subcutaneous Hepa 1-6 liver cancer models in C57BL/6 mice.

Cell Culture: The Hepa 1-6 tumor cells were maintained in vitro as a monolayer culture in DMEM medium supplemented with 10% fetal bovine serum at 37° C. in an atmosphere of 5% CO2 in air. The cells in an exponential growth phase were harvested and counted for tumor inoculation.

Tumor Inoculation: Each C57BL/6 mouse was subcutaneously inoculated at the right flank with Hepa 1-6 tumor cells (5×10⁶) in 0.1 ml of PBS for tumor development. The date of tumor cell inoculation was denoted as day 0.

Randomization: The randomization started when the mean tumor size reached approximately 100-150 mm³ in each model. 120 mice were enrolled in the study. All animals were randomly allocated to 10 study groups by tumor volume and ensure there was no significant difference in body weights between groups. Randomization was performed based on randomized block design.

After tumor cell inoculation, the animals were checked daily for morbidity and mortality. During routine monitoring, the animals were checked for any effects of tumor growth and treatments on behavior such as mobility, food and water consumption, body weight gain/loss (body weights will be measured twice per week after randomization), eye/hair matting and any other abnormalities. Mortality and observed clinical signs were recorded for individual animals in detail.

Tumor volumes were measured twice per week in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. Table 10 below shows the administration schedule for the various testing groups.

TABLE 10
Administration schedule for various testing groups.

| | | | Dosing/Administration | | | | |
|---|---|---|---|---|---|---|---|
| | | | Dose level | Sol. Conc. | Vol. | | Frequency & |
| Grp. | No. | Treatment | (mg/kg) | (mg/ml) | (μL/g) | Route | Duration |
| 1 | 12 | Vehicle | — | — | 10 | p.o. | QD × 5 days |
| 2 | 12 | Tucaresol | 10 | 1 | 10 | p.o. | QD × 5 days |
| 3 | 12 | Tucaresol | 50 | 5 | 10 | p.o. | QD × 5 days |
| 4 | 12 | Compound | 50 | 5 | 10 | p.o. | QD × 5 days |
| | | C02 | | | | | |
| 5 | 12 | Compound | 100 | 10 | 10 | p.o. | QD × 5 days |
| | | C02 | | | | | |
| 6 | 12 | Vehicle | — | | 10 | p.o. | Q2D × 10 |
| 7 | 12 | Tucaresol | 10 | 1 | 10 | p.o. | Q2D × 10 |
| 8 | 12 | Tucaresol | 50 | 5 | 10 | p.o. | Q2D × 10 |
| 9 | 12 | Compound | 50 | 5 | 10 | p.o. | Q2D × 10 |
| | | C02 | | | | | |
| 10 | 12 | Compound | 100 | 10 | 10 | p.o. | Q2D × 10 |
| | | C02 | | | | | |

QD: once per day; Q2D: every other day

Tumor growth inhibition (TGI): TGI % is an indication of antitumor activity, and expressed as: TGI (%)=100×(1−T/C). T and C are the mean tumor volume (or weight) of the treated and control groups, respectively, on a given day. Statistical analysis of the difference in mean tumor volume among the groups were conducted using the data collected on the day when mean TV of vehicle group reaches the humane endpoints so that TGI can be derived for all/most mice enrolled in the study. ΔAUC=Statistical analysis performed with a linear mixed effect regression model. The study would be terminated when the mean tumor burden of the vehicle treated control group reaches 2,000 mm³ or one week following the final dose, whichever comes first.

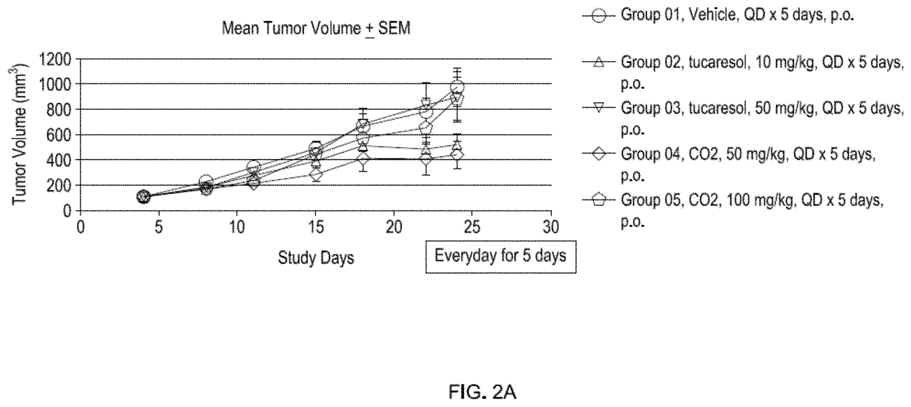

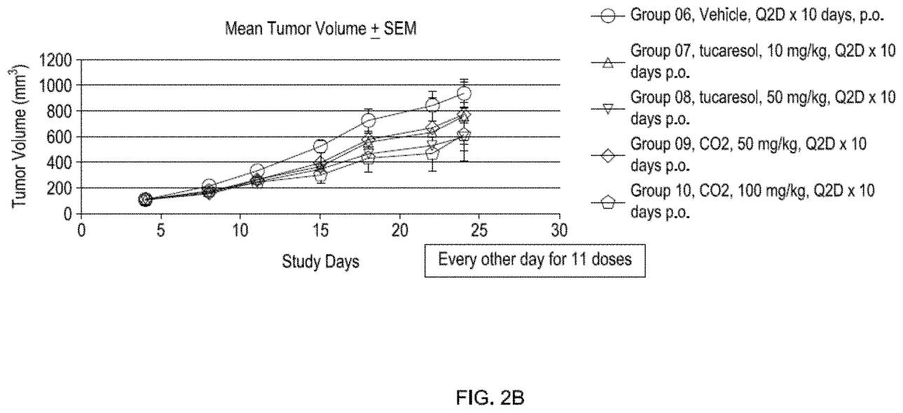

The results of tucaresol and compound C02 efficacy in murine liver cancer model are shown in FIG. 2A and 2B. FIG. 2A shows the treatment results of group 1 to group 5, and FIG. 2B shows the results of group 6 to group 10. The results indicated that compound C02 and tucaresol can achieve similar efficacy with orally administered C02 achieving greater efficacy. Administration of Compound C02 once a day for five days (QD×5) achieved greater efficacy than every-other-day administration. Once efficacy was achieved, no change in the high level immune cell infiltration of the tumor was detected.

### EXAMPLE 9

### In Vivo Efficacy Study for Compound C02 as a Single Agent and in Combination with Anti-PD-1 antibody in the Treatment of Subcutaneous MC-38 Murine Colorectal Cancer Model

This study evaluated preclinically the in vivo therapeutic efficacy of Compound C02 as a single agent and in combination with anti-PD-1 antibody in the treatment of subcutaneous MC-38 colorectal cancer models in C57BL/6 mice. Table 11 below shows the administration schedule for various groups studied.

TABLE 11
Administration schedule for treatment groups.

| | | | Dosing/Administration | | | | |
|---|---|---|---|---|---|---|---|
| | | | Dose level | Sol. Conc. | Vol. | | Frequency & |
| Grp. | No. | Treatment | (mg/kg) | (mg/ml) | (μL/g) | Route | Duration |
| 1 | 12 | Control IgG | 10 | 1 | 10 | i.p. | BIW |
| | | Vehicle for C02 | — | — | 10 | p.o. | QD × 5 days |
| 2 | 12 | Anti-PD1 (RMP 1-14) | 10 | 1 | 10 | i.p. | BIW |
| | | Vehicle for C02 | — | — | 10 | p.o. | QD × 5 days |
| 3 | 12 | Control IgG | 10 | 1 | 10 | i.p. | BIW |
| | | C02 | 10 | 1 | 10 | p.o. | QD × 5 days |
| 4 | 12 | Control IgG | 10 | 1 | 10 | i.p. | BIW |
| | | C02 | 25 | 2.5 | 10 | p.o. | QD × 5 days |
| 5 | 12 | Control IgG | 10 | 1 | 10 | i.p. | BIW |
| | | C02 | 50 | 5 | 10 | p.o. | QD × 5 days |
| 6 | 12 | Control IgG | 10 | 1 | 10 | i.p. | BIW |
| | | C02 | 100 | 10 | 10 | p.o. | QD × 5 days |
| 7 | 12 | Anti-PD1 | 10 | 1 | 10 | i.p. | BIW |
| | | C02 | 10 | 1 | 10 | p.o. | QD × 5 days |
| 8 | 12 | Anti-PD1 | 10 | 1 | 10 | i.p. | BIW |
| | | C02 | 25 | 2.5 | 10 | p.o. | QD × 5 days |
| 9 | 12 | Anti-PD1 | 10 | 1 | 10 | i.p. | BIW |
| | | C02 | 50 | 5 | 10 | p.o. | QD × 5 days |
| 10 | 12 | Anti-PD1 | 10 | 1 | 10 | i.p. | BIW |
| | | C02 | 100 | 10 | 10 | p.o. | QD × 5 days |

BIW: twice a week; QD: once a day

Treatment regimen may be changed per BW loss or other adverse effect according to rules set forth and/or client requests. When dosed on the same day, vehicle for CO2 was administered about 1-2 hours after the dosing of anti-PD1 or control IgG.

The Hepa 1-6 tumor cells were maintained in vitro as a monolayer culture in RPMI-1640 supplemented with 10% fetal bovine serum at 37° C. in an atmosphere of 5% CO2 in air. The cells in an exponential growth phase were harvested and counted for tumor inoculation.

Tumor Inoculation: Each C57BL/6 mouse were subcutaneously inoculated at the right flank with MC-38 tumor cells (1×10⁶) in 0.1 ml of PBS for tumor development. The date of tumor cell inoculation was denoted as day 0.

Randomization: The randomization started when the mean tumor size reaches approximately 100-150 mm³ in each model. 120 mice were enrolled in the study. All animals were randomly allocated to 10 study groups by tumor volume and ensure there was no significant difference in body weights between groups. Randomization was performed based on randomized block design. The treatment for Vehicle for C02, anti-PD1 and control IgG were started immediately after randomization. The date of randomization and first treatment was denoted as PG (post grouping)-day 0.

After tumor cells inoculation, the animals were checked daily for morbidity and mortality. During routine monitoring, the animals were checked for any effects of tumor growth and treatments on behavior such as mobility, food and water consumption, body weight gain/loss (body weights will be measured twice per week after randomization), eye/hair matting and any other abnormalities. Mortality and observed clinical signs were recorded for individual animals in detail.

Tumor volumes were measured twice per week in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

Rectal temperature was recorded without anaesthesia on the day prior to the first dosing and on the day after the last oral dose.

The administration schedule in the various testing groups are shown in Table 11 below.

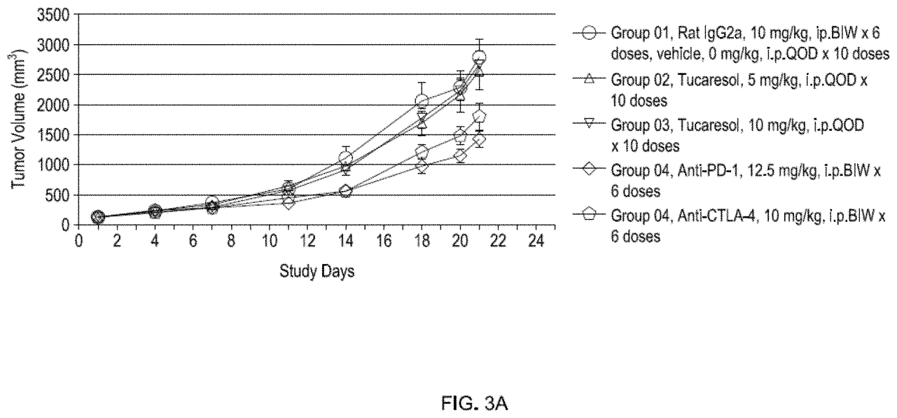

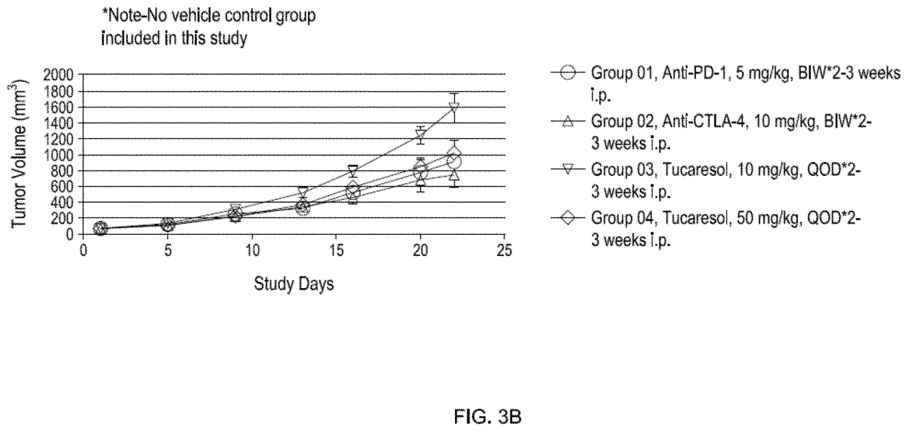

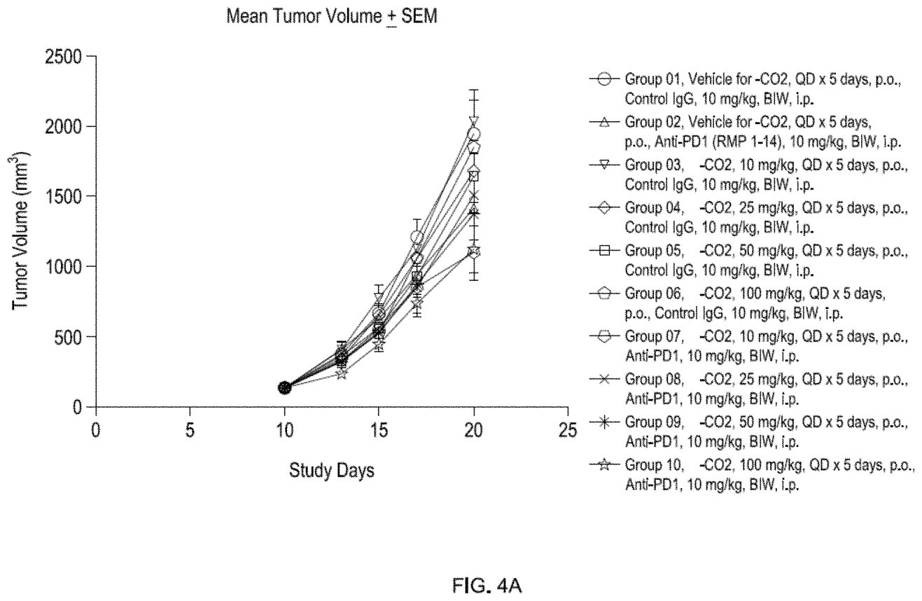

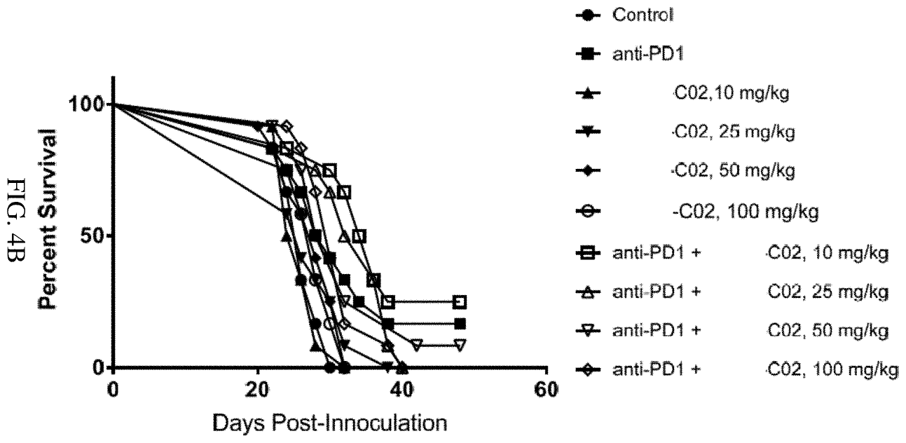

FIG. 3A and FIG. 3B show the inhibition activity of tucaresol, anti-PD1, and anti-CTLA4 groups at various doses in MC38 Murine Colorectal Cancer model. FIG. 4A and FIG. 4B respectively show the tumor growth and survival rate of Compound 02 and anti-PD1 combination at various doses in MC38 Murine colorectal cancer model. The established benefits of administration once a day for five days (QDx5) compound C02 as a monotherapy or in combination with anti-PD1 were especially apparent after about 15-20 days of treatment, indicating an immunologic MOA (e.g. boosting existing immune responses or neoantigen generation). The efficacy of QDx5 dosing of compound C02 exhibited an inverse dose response relationship, even down to 10 mg/kg QDx5.

### Example 10

### In Vivo study of C02 in Combination with Anti-PD1 in the Treatment of Subcutaneous Syngeneic Colorectal Cancer Model MC38 in Female C47BL/6 Mice

MC38 tumor cells were maintained in vitro with DMEM medium supplemented with 10% fetal bovine serum at 37° C. in an atmosphere of 5% CO₂ in air. The cells in exponential growth phase were harvested and quantitated by cell counter before tumor inoculation. Each mouse was inoculated subcutaneously in the right lower flank with 1×10⁶ MC38 tumor cells in 0.1 ml of PBS for tumor development.

The randomization was started when the mean tumor size reached approximately 145 mm³. 108 mice were enrolled in the study. All animals were randomly allocated to 9 study groups. Randomization by tumor volume was performed based on “Matched distribution” method/ “Stratified” method (StudyDirector™ software, version 3.1.399.19)/randomized block design.

After tumor cell inoculation, the animals were checked daily for morbidity and mortality. During routine monitoring, the animals were checked for any effects of tumor growth and treatments on behavior such as mobility, food and water consumption, body weight gain/loss (Body weights were measured three times a week after randomization), eye/hair matting and any other abnormalities. Mortality and observed clinical signs were recorded for individual animals in detail.

Tumor volumes were measured three times a week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using StudyDirector™ software (version 3.1.399.19).

The treatment was initiated following randomization. The test article administration and the animal numbers in the study groups described here were as shown in Table 12.

TABLE 12
Treatment Groups

| | | | Dose | Conc. | Vol. | | Frequency and |
|---|---|---|---|---|---|---|---|
| Group | No. | Treatment | (mg/kg) | (mg/ml) | (μL/g) | ROA | Duration |
| 1 | 12 | Vehicle | | | 10 | p.o. | QD × 5 (day 1~5) |
| | | Rat IgG2a | 10 | 1 | 10 | i.p. | BIW × 6 doses (day |
| | | | | | | | 1, 4, 8, 11, 15, 18) |
| | | Vehicle | | | 10 | i.p. | Q9D × 2 weeks |
| | | | | | | | (day 1, day 10) |
| 2 | 12 | C02 | 20 | 2 | 10 | p.o. | QD × 5 (day 1~5) |
| | | Rat IgG2a | 10 | 1 | 10 | i.p. | BIW × 6 doses (day |
| | | | | | | | 1, 4, 8, 11, 15, 18) |
| | | Vehicle | | | | i.p. | Q9D × 2 weeks |
| | | | | | | | (day 1, day 10) |
| 3 | 12 | Vehicle | | | 10 | p.o. | QD × 5 (day 1~5) |
| | | PD-1 | 10 | 1 | 10 | i.p. | BIW × 6 doses (day |
| | | (RMP 1-14) | | | | | 1, 4, 8, 11, 15, 18) |
| | | Vehicle | | | 10 | i.p. | Q9D × 2 weeks |
| | | | | | | | (day 1, day 10) |
| 4 | 12 | C02 | 20 | 2 | 10 | p.o. | QD × 5 (day 1~5) |
| | | PD-1 | 10 | 1 | 10 | i.p. | BIW × 6 doses (day |
| | | (RMP 1-14) | | | | | 1, 4, 8, 11, 15, 18) |
| | | Vehicle | | | 10 | i.p. | Q9D × 2 weeks |
| | | | | | | | (day 1, day 10) |

After one measurement of body weight loss (BWL)>20% or BWL>15% for consecutive 72 hours, the individual mouse was sacrificed. BWL was calculated based on the BW of mouse on the first day of treatment. After one measurement of BWL>15%, dosing holidays were given to the individual mouse with BWL>15% which was housed in a separate cage alone with daily monitoring for 72 hours, while other mice in the same group would receive dosing as scheduled., treatment was resumed when the BWL has recovered to BWL<10%. Supplemental gel was supplied to all the animals in the study on day 5 to day 15 upon sponsor's request.

Tumor growth inhibition (TGI): TGI% is an indication of antitumor activity, and expressed as: TGI (%)=100×(1−T/C). T and C are the mean tumor volume (or weight) of the treated and control groups, respectively, on a given day. Statistical analysis of the difference in mean tumor volume among the groups was conducted using the method time to survival event/euthanasia. Individual mice were euthanized when tumor volume exceeded 3000mm³. The study was ultimately terminated at Day 36.

The body weight of all animals was monitored throughout the study and animals were euthanized if they lost over 15% of their body weight relative to the weight at the first day of treatment for 3 consecutive days, or lose over 20% of their body weight relative to the weight at the first day of treatment.

Individual mouse with tumor volume exceeded 3000 mm³ (Sacrifice the individual mouse). Mouse with tumor ulceration (Hair loss at the reddened site, wound exudates (wet open wound, bleeding) and/or skin is no longer closed (pitted, concave, no scab) was euthanized. Severe dehydration, hypothermia, abnormal/labored respiration, lethargy, obvious pain, diarrhea, skin lesions, neurological symptoms, impaired mobility (not able to eat or drink) due to significant ascites and enlarged abdomen, astasia, continuous prone or lateral position, signs of muscular atrophy, paralytic gait, clonic convulsions, tonic convulsions, persistent bleeding from body orifice.

To compare tumor volumes of different groups at a pre-specified day, Bartlett's test was used to check the assumption of homogeneity of variance across all groups. When the p-value of Bartlett's test was >=0.05, one-way ANOVA was runto test overall equality of means across all groups. If the p-value of the one-way ANOVA was <0.05, was performed post hoc testing by running Tukey's HSD (honest significant difference) tests for all pairwise comparisons, and Dunnett's tests for comparing each treatment group with the vehicle group. When the p-value of Bartlett's test was <0.05, was run Kruskal-Wallis test to test overall equality of medians among all groups. If the p-value the Kruskal-Wallis test was <0.05, was performed post hoc testing by running Conover's non-parametric test for all pairwise comparisons or for comparing each treatment group with the vehicle group, both with single-step p-value adjustment.

In addition, pairwise comparisons were performed without multiple comparison correction and reported nominal/uncorrected p-values directly from Welch's t-test or Mann-Whitney U test. Specifically, Bartlett's test was used to check the assumption of homogeneity of variance for a pair of groups. When the p-value of Bartlett's test was >0.05, Welch's t-test was run, otherwise Mann-Whitney U test was run, to obtain nominal p-values. Survival curves were compared between groups by logrank (Mantel-Cox) test.

All statistical analyses had been done in R-a language and environment for statistical computing and graphics (version 3.3.1). All tests were two-sided unless otherwise specified, and p-values of <0.05 were regarded as statistically significant.

Kaplan-Meier plot showing percent survival of mice treated with G1 to G9. A mouse whose tumor volume exceeded 3000 mm³ was sacrificed and scored in the Kaplan-Meier plot. Tables 13.1 and 13.2 below describes the tumor growth inhibition with data collected over 24 days. Tables 14.1 and 14.2 below describes mean body weight.

TABLE 13.1
Mean Tumor Volumes from Days 1 to 12

| Group | 1 | 4 | 8 | 10 | 12 |
|---|---|---|---|---|---|
| Group 01 | 145.01 ± 5.39 | 252.75 ± 19.69 | 613.84 ± 79.51 | 1150.61 ± 144.3 | 1787.72 ± 219.81 |
| Group 02 | 145 ± 5.46 | 215.49 ± 15.14 | 590.08 ± 59.54 | 1048.2 ± 89.19 | 1750.55 ± 154.11 |
| Group 03 | 145 ± 5.32 | 235.91 ± 17.87 | 642.95 ± 76.3 | 1180.55 ± 167.54 | 2089 ± 230.17 |
| Group 04 | 144.97 ± 6.74 | 215.06 ± 14.64 | 560.59 ± 57.84 | 888.86 ± 107.12 | 1430.43 ± 173.12 |

TABLE 13.2
Mean Tumor Volumes from Days 15 to 24

| Group | 15 | 17 | 19 | 22 | 24 |
|---|---|---|---|---|---|
| Group 01 | NA | NA | NA | NA | NA |
| Group 02 | 2847.19 ± 242.97 | NA | NA | NA | NA |
| Group 03 | NA | NA | NA | NA | NA |
| Group 04 | 2274.32 ± 343.76 | NA | NA | NA | NA |

TABLE 14.1
Mean Body Weight from Days 1 to 12

| Group | 1 | 4 | 8 | 10 | 12 |
|---|---|---|---|---|---|
| Group 01 | 18.8 ± 0.2 | 19.4 ± 0.2 | 19.9 ± 0.3 | 20.3 ± 0.3 | 21.1 ± 0.3 |
| Group 02 | 18.9 ± 0.2 | 19.2 ± 0.2 | 19.6 ± 0.2 | 20 ± 0.3 | 20.6 ± 0.4 |
| Group 03 | 19 ± 0.3 | 19.3 ± 0.3 | 19.9 ± 0.3 | 20.7 ± 0.4 | 21.7 ± 0.5 |
| Group 04 | 19.2 ± 0.2 | 19.3 ± 0.2 | 19.9 ± 0.1 | 20.3 ± 0.2 | 21 ± 0.2 |

TABLE 14.2
Mean Body Weight from Days 15 to 24

| Group | 15 | 17 | 19 | 22 | 24 |
|---|---|---|---|---|---|
| Group 01 | NA | NA | NA | NA | NA |
| Group 02 | 22.3 ± 0.5 | NA | NA | NA | NA |
| Group 03 | NA | NA | NA | NA | NA |
| Group 04 | 21.9 ± 0.4 | NA | NA | NA | NA |

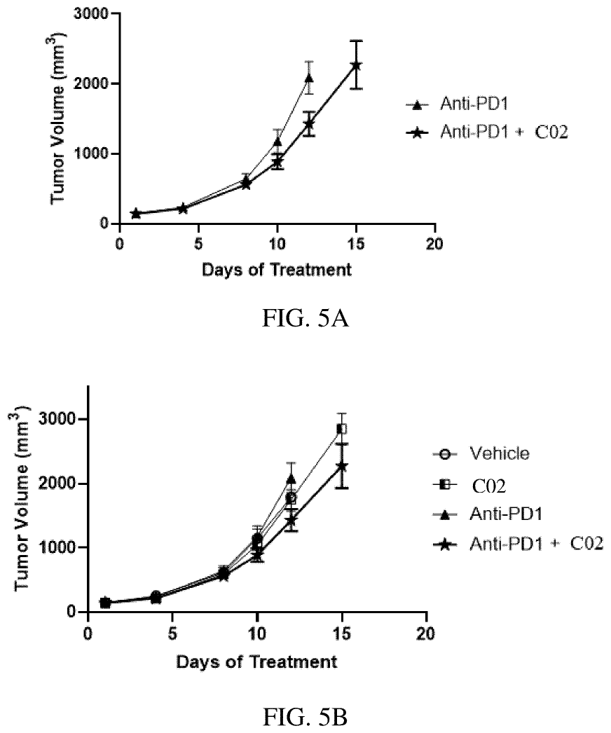

With the methods utilized, C02 (20 mg/kg, PO, QDx 5 (dayl-5)) was not associated with weight loss in the MC38 murine colorectal cancer model established in immunocompetent mice, as a monotherapy or in combination with anti-PD1. FIGS. 5A and 5B graphically illustrates the tumor volumes over time. As shown in FIG. 5A,the 20 mg/kg C02 (Group 4) significantly increased the antitumor efficacy of anti-PDA1 administered alone (Group 3).

What is claimed is:

1. A compound having a structure of formula (I): 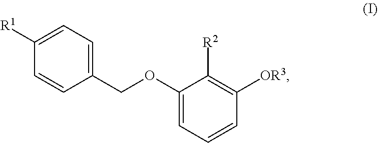
wherein:
R¹ is —COOH, —COOR^{1a}, —COO(CH₂)ₘC(O)NR^{1a}R^{2a}, —CONHR^{1b}, —COR⁴, or —CONH(CH₂)ₘCOOR^{2b};
R² is —CH(O) or —CH(═NOR^{1a});
R³ is H, —C(O)R^{1a}, optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl;
R⁴ is an amino acid residue attached through an N-terminal amine;
each R^{1a}, R^{2a}, R^{1b} and R^{2b} are independently selected from—H, halogen,—OH, —COOH,—COO(C₁₋₄ alkyl), optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl; and
m is an integer between 0 to 3;
or a pharmaceutically acceptable salt thereof.

2. The compound of claim 1, wherein the compound has a structure of formula (II) 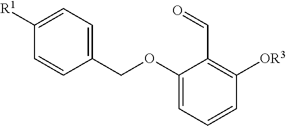
or a pharmaceutically acceptable salt thereof.

3. The compound of claim 1, wherein the compound has a structure of formula (III) 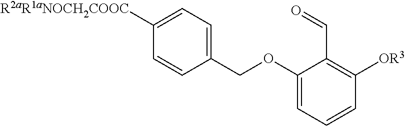
or a pharmaceutically acceptable salt thereof.

4. The compound of claim 1, wherein R¹ is —CONHR^{1b} and R^{1b} is an optionally substituted 3-8 membered heterocyclyl, or an optionally substituted 5-6 membered monosaccharide ring.

5. The compound of claim 4, wherein R^{1b} is a deoxyglucose.

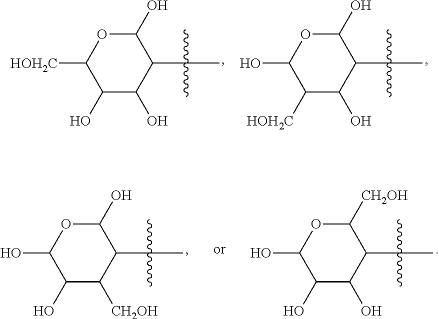 [SMILES: *C1C(O)OC(CO)C(O)C1O ; *C1C(O)OC(O)C(CO)C1O ; *C1C(O)OC(O)C(O)C1CO ; *C1C(CO)OC(O)C(O)C1O]

6. The compound of claim 4 or 5, wherein R^{1b} is

7. The compound of claim 1, wherein R² is —CH(═NOR^{1a}).

8. The compound of claim 7, wherein R² is —CH(═NOH).

9. The compound of claim 7, wherein R^{1a}is a C₁₋₆ alkyl.

10. The compound of claim 9, wherein R^{1a}is ethyl, butyl, cetyl, decyl or dodecyl

11. The compound of claim 1, wherein R¹ is—COR⁴.

12. The compound of claim 11, wherein R⁴ is a L-amino acid residue.

13. The compound of claim 11 or 12, wherein R⁴ is a L-Lys or L-Glu residue.

14. The compound of claim 11 or 12, wherein R⁴ is an amino acid residue selected from Gly, Ala, Phe, Tyr, Glu, Leu, Ser, Arg, Gln, Val, Lys, Thr, Asn, Met, Cys, Trp, Asp, His, Pro, or Ile.

15. The compound of claim 1, wherein R¹ is —CONHCH(COOH)(CH₂)ₙR⁵, n is an integer between 0 to 5, and R⁵ is selected from —H, —OH, —COOH, —COO(C₁₋₄ alkyl), —NHC(═NH)NH_{2,} optionally substituted C₁₋₁₀ alkyl, optionally substituted C₂₋₁₀ alkenyl, optionally substituted C₂₋₁₀ alkynyl, optionally substituted C₃₋₇ cycloalkyl, optionally substituted 3-8 membered heterocyclyl, optionally substituted 5-6 membered monosaccharide ring, optionally substituted C₆₋₁₀ aryl, or optionally substituted 5-10 membered heteroaryl.

16. The compound of claim 15, wherein n is an integer between 0 and 3.

17. The compound of claim 15 or 16, wherein R⁵ is H, methyl, phenyl, 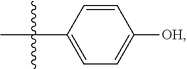 [SMILES: *c1ccc(O)cc1]
—COOH, —CH(CH₃)₂, —OH, —OCH₃, —NHC(═NH)NH₂, —CONH₂,—CH(CH₃)₂, —NH₂,—SH, —SCH₃, —CH(CH₃)(CH₂OH), indolyl, —CH(CH₃)(CH₂CH₃), imidazolyl, or pyrrolidinyl.

18. The compound of claim 1, wherein R³ is acyl, D-glucose, 2-deoxy-D-glucose, D-ribose, or 2-deoxy-D-ribose.

19. The compound of claim 18, wherein R³ is

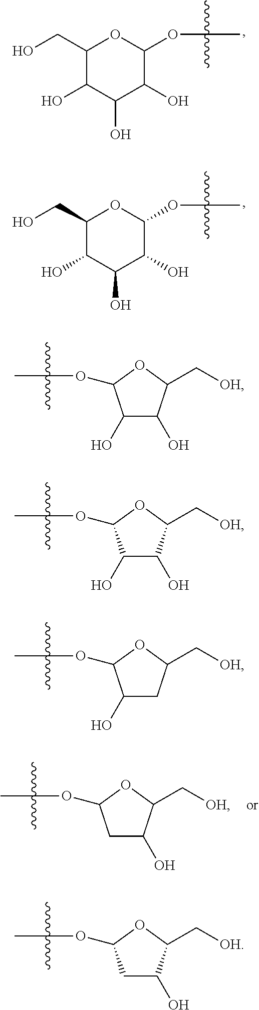

20. The compound of claim 1, wherein the compound is selected from 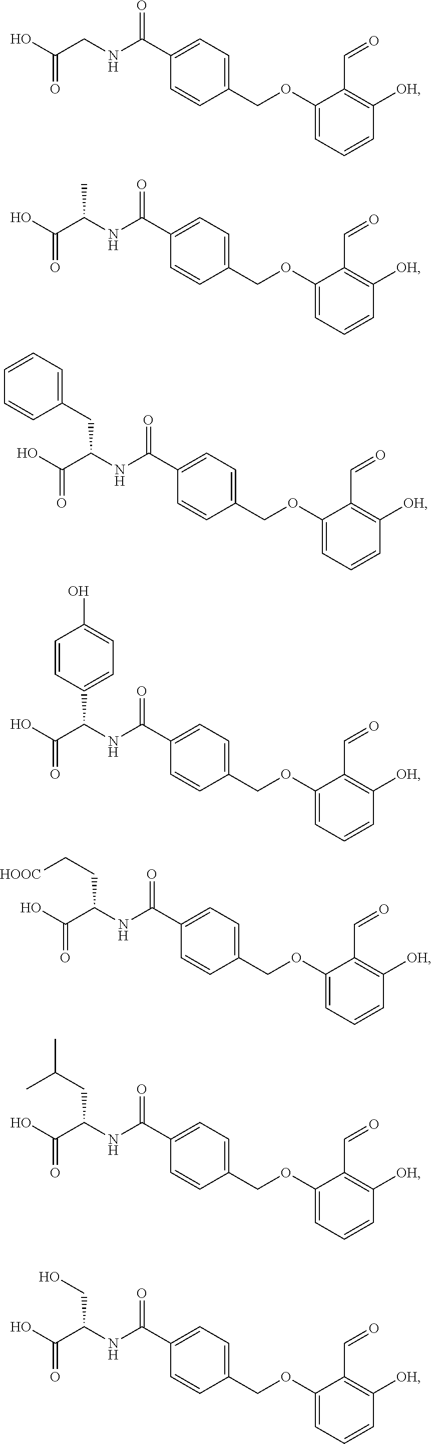 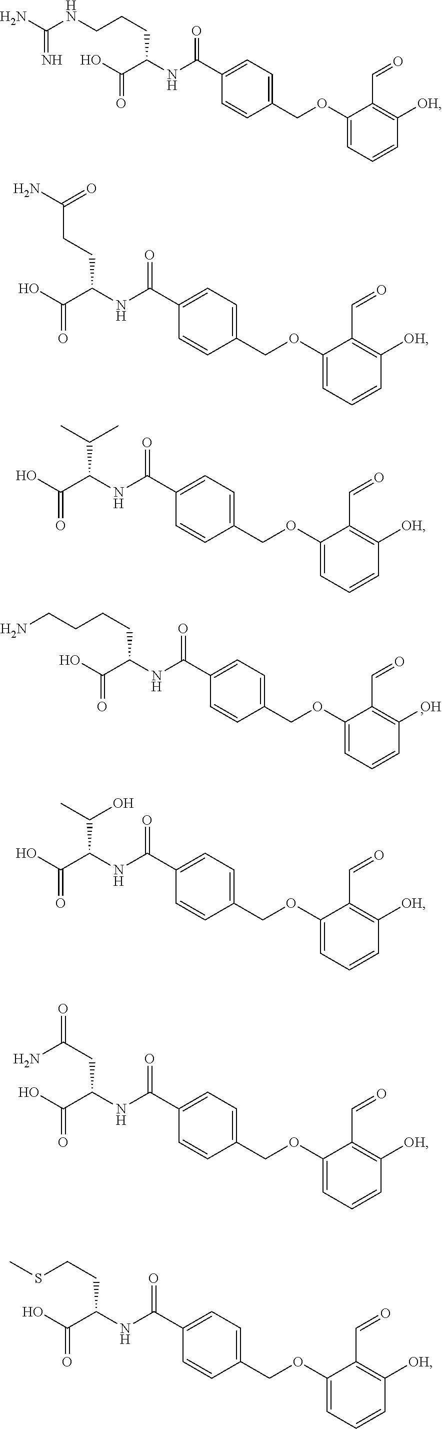 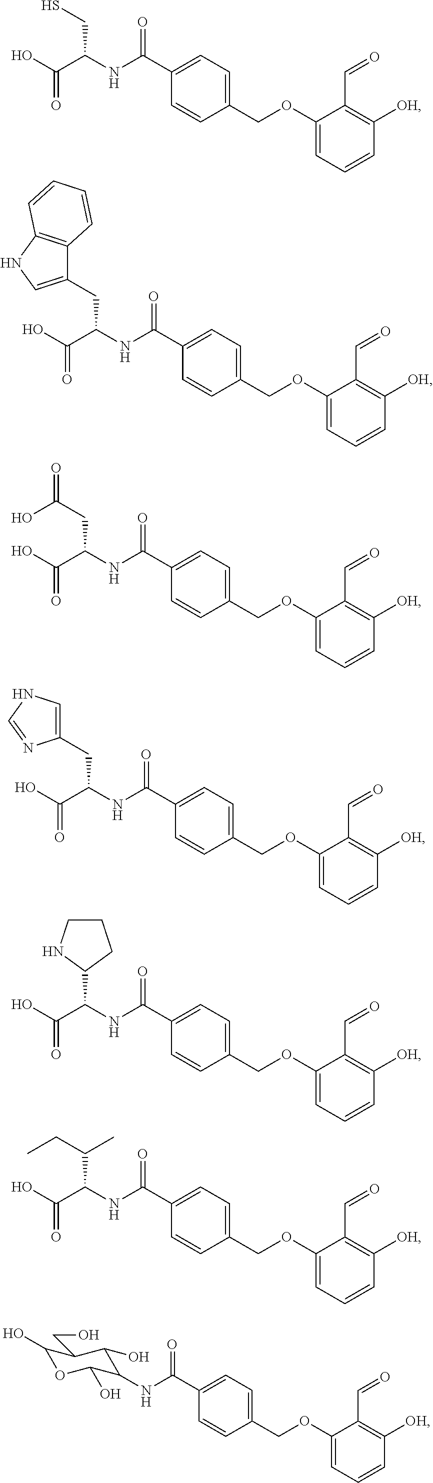 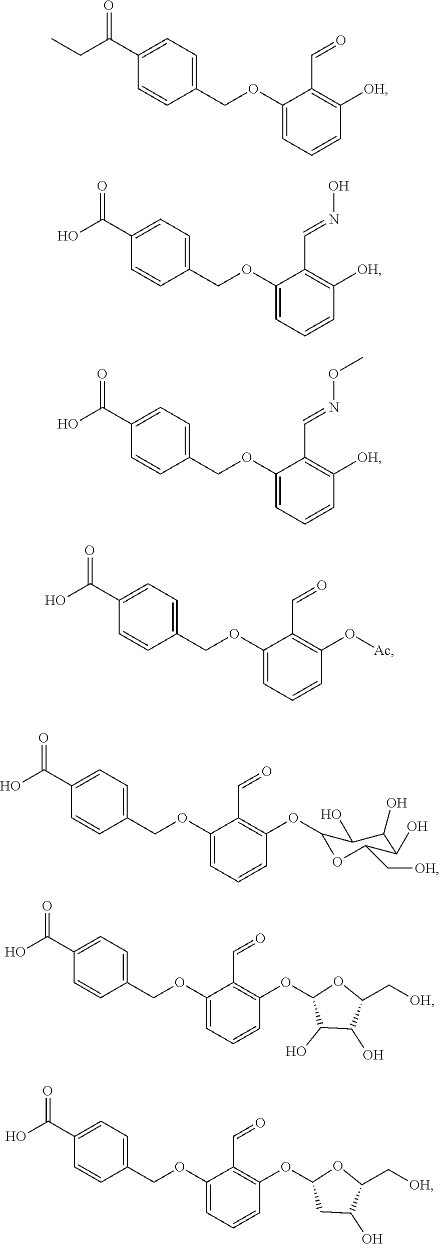
or pharmaceutically acceptable salts thereof.

21. A pharmaceutical composition comprising a compound or pharmaceutically acceptable salts thereof of any one of the preceding claims and at least a pharmaceutically acceptable carrier or excipient.

22. The pharmaceutical composition of claim 21, further comprising one or more additional agents.

23. The pharmaceutical composition of claim 21 or 22, further comprising one or more immune checkpoint inhibitors.

24. The pharmaceutical composition of claim 23, wherein each of the one or more immune checkpoint inhibitors is independently an inhibitor of PD-1, PD-L1, PD-L2, PD-L3, PD-L4, CTLA-4, LAG3, B7-H3, B7-H4, KIR or TIM3.

25. The pharmaceutical composition of any one of claims 21 to 24, further comprising plinabulin.

26. The pharmaceutical composition of any one of claims 21 to 25, further comprising one or more additional chemotherapeutic agent.

27. The pharmaceutical composition of any one of claims 21 to 26, further comprising one or more pharmaceutically acceptable excipients.

28. Use of the compound or a pharmaceutically acceptable salt thereof of any one of claims 1 to 20 in the manufacture of a medicament for treating cancer in a subject.

29. Use of the compound or a pharmaceutically acceptable salt thereof of any one of claims 1 to 20 in the manufacture of a medicament for enhancing immune response in a subject.

30. Use of the compound or a pharmaceutically acceptable salt thereof of any one of claims 1 to 20 or a pharmaceutical composition of any one of claims 21 to 27 in the manufacture of a medicament for enhancing immune response in a cancer subject.

31. The use of any one of claims 28-30, wherein the subject is administered a vaccine or immunization.

32. The use of any one of claims 28-31, wherein the subject is administered one or more additional agents.

33. The use of any one of claims 28-32, wherein the subject is administered one or more immune checkpoint inhibitors.

34. The use of claim 33, wherein the immune checkpoint inhibitor is an inhibitor of PD-1, PD-L1, PD-L2, PD-L3, PD-L4, CTLA-4, LAGS, 3, B7-H3, B7-H4, KIR or TTM3 .

35. The use of any one of claims 28-34, wherein the subject is administered plinabulin.

36. The use of any one of claims 28-35, wherein the subject is administered one or more additional chemotherapeutic agent.

37. The use of any one of claims 28-36, wherein the subject is administered a radiation therapy.

38. A method of making a compound of Formula (II-A), comprising reacting a compound of formula (A-1) R—H with a compound of formula A-2 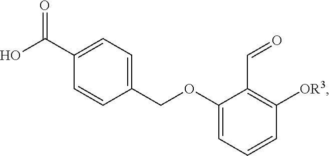
wherein R—H is an amino acid, D-glucosamine, alcohol, or hydroxylamine; R is a radical formed upon deprotonation of R—H; and R³ is H or C₁₋₆ alkyl.

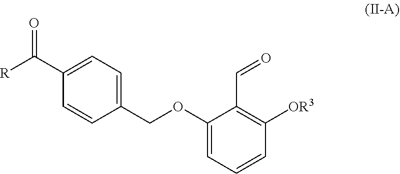

39. The method of claim 38, wherein R—H is an amino acid selected from Gly, Ala, Phe, Tyr, Glu, Leu, Ser, Arg, Gln, Val, Lys, Thr, Asn, Met, Cys, Trp, Asp, His, Pro, or Ile.

40. The method of claim 38, wherein R—H is D-glucosamine, methanol, or hydroxylamine

41. The method of claim 38, wherein R³ is H.

42. A method of making a compound of Formula (II-B), comprising reacting tucaresol with NH₂—OR^{a}, wherein R^{a} is H or C₁₋₆ alkyl.

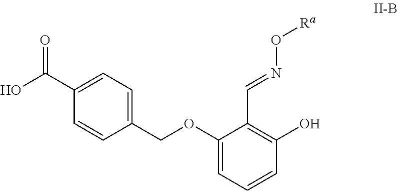

43. A method of preparing a compound of formula (II-C), comprising:
protecting the carboxylic acid group on tucaresol to form a compound of formula (A-3), and 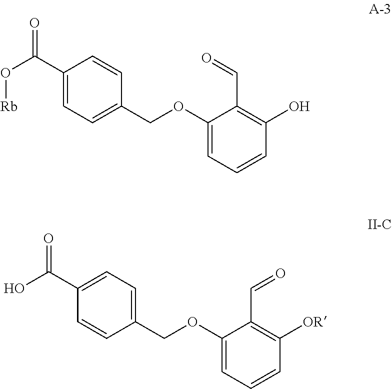
converting the hydroxyl group in the compound of formula (A-3) to an ester and then undergoing hydrolysis to form the compound of formula (II-C), wherein R′ is acyl, D-glucose, 2-deoxy-D-glucose, D-ribose, or 2-deoxy-D-ribose, and Rb is a C₁₋₆ alkyl.

44. The method of claim 43, wherein R′ is

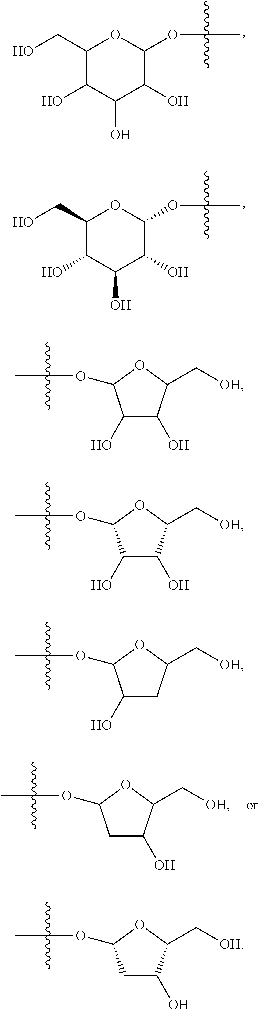

45. The method of claim 43 or 44, wherein Rb is a methyl.
